(19) United States
(12) Patent Application Publication
(10) Pub. No.: US 2013/0023177 A1
(43) Pub. Date: Jan. 24, 2013

(54) BICOMPONENT FIBERS

(71) Applicants: Gert J. Claasen, Richterswil (CH); Angels Domenech, La Selva Del Camp (ES); Thor Gudmundsson, Houston, TX (US)

(72) Inventors: Gert J. Claasen, Richterswil (CH); Angels Domenech, La Selva Del Camp (ES); Thor Gudmundsson, Houston, TX (US)

(73) Assignee: Dow Blobal Technologies LLC, Midland, MI (US)

(21) Appl. No.: 13/635,950

(22) PCT Filed: Feb. 2, 2011
(86) PCT No.: PCT/US11/23452

§ 371 Date: Sep. 19, 2012

Related U.S. Application Data
(60) Provisional application No. 61/315,435, filed on Mar. 19, 2010.

(51) Int. Cl. D01F 8/04; D01F 8/12; D04H 13/00; D04H 3/00; A61L 15/24; A61L 15/26; A41D 1/00; D02G 3/38; D01F 8/14; A47L 13/16

(52) U.S. Cl. 442/361; 428/373; 428/361; 15/209.1; 604/372; 2/69; 264/103

(57) ABSTRACT

The present invention provides bicomponent fibers, a method of producing bicomponent fibers, nonwoven materials comprising one or more such bicomponent fibers, and a method for making such nonwoven materials. The bicomponent fibers according to the present invention comprise (a) a first component comprising a polymeric material selected from the group consisting of polypropylene, polyester, and polyamide; and (b) a second component comprising a polyethylene composition comprising less than or equal to 100 percent by weight of the units derived from ethylene; and less than 20 percent by weight of units derived from one or more α-olefin comonomers; wherein the polyethylene composition has a density in the range of from 0.945 to 0. 965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition.

### CROSS-REFERENCE TO RELATED APPLICATIONS

This application is a non-provisional application claiming priority from the U.S. Provisional Patent Application No. 61/315,435, filed on Mar. 19, 2010, entitled “BICOMPONENT FIBERS,” the teachings of which are incorporated by reference herein, as if reproduced in full hereinbelow.

### FIELD OF INVENTION

The present invention relates to bicomponent fibers, method of producing bicomponent fibers, nonwoven materials comprising one or more such bicomponent fibers, and a method for making such nonwoven materials.

### BACKGROUND OF THE INVENTION

The use of polymeric compositions such as polyolefins in producing fibers is generally known. Exemplary polyolefins include, but are not limited to, polypropylene compositions. Such fibers may be formed into fabrics, e.g. non-woven fabrics. Different techniques may be employed to form such fabrics. Such techniques are generally known to persons of ordinary skill in the art.

In general, there is a correlation between polymer viscosity and the tensile properties of the spunbond web, i.e. decreasing viscosity has a negative influence in tensile properties of the spunbond web. A decrease in viscosity facilitates a decrease in system pressure; thus, providing higher throughput.

Despite the research efforts in developing composite fibers such as bicomponent fibers, there is still a need for bicomponent fibers with improved properties. Furthermore, there is still a need for a process for producing such bicomponent fibers having improved properties. Furthermore the present invention provides a decrease in viscosity of the polymeric materials while improving and/or maintaining mechanical properties of the formed thermoboanded webs. The reduction in polymer viscosity leads to lower shear rates; and hence, higher throughputs can be achieved. The lower shear rates in the system facilitate an increase in the lifetime of the spin pack; thereby, providing improved bicomponent spunbond fibers.

### SUMMARY OF THE INVENTION

The present invention provides bicomponent fibers, a method of producing bicomponent fibers, nonwoven materials comprising one or more such bicomponent fibers, and a method for making such nonwoven materials.

In one embodiment, the instant invention provides a bicomponent fiber comprising (a) a first component comprising a polymeric material selected from the group consisting of polypropylene, polyester, and polyamide; and (b) a second component comprising a polyethylene composition comprising less than or equal to 100 percent by weight of the units derived from ethylene; and less than 20 percent by weight of units derived from one or more α-olefin comonomers; wherein the polyethylene composition has a density in the range of from 0.945 to 0.965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition.

In an alternative embodiment, the instant invention further provides a method for producing a bicomponent fiber comprising the steps of: (1) selecting a first component comprising a polymeric material selected from the group consisting of polypropylene, polyester, and polyamide; (2) selecting a second component comprising a polyethylene composition comprising less than or equal to 100 percent by weight of the units derived from ethylene; and less than 20 percent by weight of units derived from one or more α-olefin comonomers; wherein said polyethylene composition has a density in the range of from 0.945 to 0.965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition; (3) spinning said first component and said second component into a bicomponent fiber; and (4) thereby forming said bicomponent fiber.

In an alternative embodiment, the instant invention further provides a nonwoven material comprising one or more bicomponent fibers, as described above.

In an alternative embodiment, the instant invention further provides a process for fabricating a spunbond fabric comprising the steps of: (1) providing one or more bicomponent fibers, as described hereinabove, (2) spunbonding said one or more bicomponent fibers; and (3) thereby forming said spunbond fabric.

In an alternative embodiment, the instant invention provides a bicomponent fiber, method of producing the same, nonwoven materials made therefrom, and method of making such nonwoven materials, in accordance with any of the preceding embodiments, except that the bicomponent fiber has a denier per filament in the range of from 0.1 to 50 g/9000 m.

In an alternative embodiment, the instant invention provides a bicomponent fiber, method of producing the same, nonwoven materials made therefrom, and method of making such nonwoven materials, in accordance with any of the preceding embodiments, except that the bicomponent fiber has a denier per filament in the range of from 0.1 to 10 g/9000 m.

In an alternative embodiment, the instant invention provides a bicomponent fiber, method of producing the same, nonwoven materials made therefrom, and method of making such nonwoven materials, in accordance with any of the preceding embodiments, except that the bicomponent fiber has a denier per filament in the range of from 1.6 to 2.4 g/9000 m.

In an alternative embodiment, the instant invention provides a bicomponent fiber, method of producing the same, nonwoven materials made therefrom, and method of making such nonwoven materials, in accordance with any of the preceding embodiments, except that the nonwoven fabric has an tensile elongation in machine direction (MD) in the range of from 50 to 200 percent, measured by cutting Spunbond fabrics into 1×6 inch specimens and testing the specimens in the machine direction (MD) using an INSTRON. The specimens were tested at 8 inches/minute with 4 inches gauge. The MD extensibility was determined at the peak force.

In an alternative embodiment, the instant invention provides a bicomponent fiber, method of producing the same, nonwoven materials made therefrom, and method of making such nonwoven materials, in accordance with any of the preceding embodiments, except that the nonwoven fabric has an tensile elongation in cross direction (CD) in the range of from 50 to 250 percent, measured by cutting Spunbond fabrics into 1×6 inch specimens and testing the specimens in the cross direction (CD) using an INSTRON. The specimens were tested at 8 inches/minute with 4 inches gauge. The CD extensibility was determined at the peak force.

In an alternative embodiment, the instant invention provides a bicomponent fiber, method of producing the same, nonwoven materials made therefrom, and method of making such nonwoven materials, in accordance with any of the preceding embodiments, except that the bicomponent fiber has a core/sheath (C/S) configuration, and wherein the core comprises the first component and the sheath comprises the second component.

In an alternative embodiment, the instant invention provides a bicomponent fiber, method of producing the same, nonwoven materials made therefrom, and method of making such nonwoven materials, in accordance with any of the preceding embodiments, except that the bicomponent fiber is a continuous fiber or a staple fiber.

In an alternative embodiment, the instant invention provides a bicomponent fiber, method of producing the same, nonwoven materials made therefrom, and method of making such nonwoven materials, in accordance with any of the preceding embodiments, except that the method of producing the bicomponent fiber further comprises the step of orienting the bicomponent fiber.

In an alternative embodiment, the instant invention provides a bicomponent fiber, method of producing the same, nonwoven materials made therefrom, and method of making such nonwoven materials, in accordance with any of the preceding embodiments, except that the one or more bicomponent staple fibers are oriented via cold drawing.

In an alternative embodiment, the instant invention provides a bicomponent fiber, method of producing the same, nonwoven materials made therefrom, and method of making such nonwoven materials, in accordance with any of the preceding embodiments, except that the process for producing a bicomponent fiber further comprises the step of annealing the bicomponent fiber.

In an alternative embodiment, the instant invention provides a bicomponent fiber, method of producing the same, nonwoven materials made therefrom, and method of making such nonwoven materials, in accordance with any of the preceding embodiments, except that the annealing step of the bicomponent fiber is carried out at 70° C. or above.

In an alternative embodiment, the instant invention provides a bicomponent fiber, method of producing the same, nonwoven materials made therefrom, and method of making such nonwoven materials, in accordance with any of the preceding embodiments, except that the nonwoven fabric has an abrasion resistance in the range of less than 1 mg/cm². Abrasion resistance was measured by abrading a spunbond fabric using a Sutherland 2000 Rub Tester to determine the fuzz level. An 11.0×4.0 cm piece of non-woven spunbond fabric was abraded with 320-grit aluminum oxide sandpaper under 2 lbs weight with 20 cycles at a rate of 42 cycles per minute, which resulted in loose fibers accumulating on the top of the spunbond fabric. The loose fibers were collected using tape and measured gravimetrically.

In an alternative embodiment, the instant invention provides a bicomponent fiber, method of producing the same, nonwoven materials made therefrom, and method of making such nonwoven materials, in accordance with any of the preceding embodiments, except that the nonwoven fabric has an abrasion resistance in the range of less than 0.5 mg/cm². Abrasion resistance was measured by abrading a spunbond fabric using a Sutherland 2000 Rub Tester to determine the fuzz level. An 11.0 cm×4.0 cm piece of non-woven spunbond fabric was abraded with 320-grit aluminum oxide sandpaper under a 2 lbs weight with 20 cycles at a rate of 42 cycles per minute, which resulted in loose fibers accumulating on the top of the spunbond fabric. The loose fibers were collected using tape and measured gravimetrically.

In an alternative embodiment, the instant invention provides nonwoven materials made therefrom, and method of making such nonwoven materials, in accordance with any of the preceding embodiments, except that the nonwoven materials are used in an article selected from the group consisting of upholstery, apparel, wall covering, carpet, diaper topsheet, diaper backsheet, medical fabric, surgical wrap, hospital gown, wipe, textile, feminine hygiene and geotextile.

### BRIEF DESCRIPTION OF THE DRAWINGS

For the purpose of illustrating the invention, there is shown in the drawings a form that is exemplary; it being understood, however, that this invention is not limited to the precise arrangements and instrumentalities shown.

FIG. 1 is a graph illustrating the relationship between Tensile Strength in machine direction (MD) v. bonding temperature;

FIG. 2 is a graph illustrating the relationship between Tensile Strength in cross direction (CD) v. bonding temperature;

FIG. 3 is a graph illustrating the relationship between Tensile Elongation in machine direction (MD) v. bonding temperature;

FIG. 4 is a graph illustrating the relationship between Tensile Elongation in cross direction (CD) v. bonding temperature;

FIG. 5 is a graph illustrating the processing parameters including: pressure and torque;

FIG. 6 is a graph illustrating the processing parameters including: pressure and torque for 50/50 (core/sheath ratio).

### DETAILED DESCRIPTION OF THE INVENTION

The present invention provides bicomponent fibers, a method of producing bicomponent fibers, nonwoven materials comprising one or more such bicomponent fibers, and a method for making such nonwoven materials.

The bicomponent fibers according to the present invention comprises (a) a first component comprising a polymeric material selected from the group consisting of polypropylene, polyester, and polyamide; and (b) a second component comprising a polyethylene composition comprising less than or equal to 100 percent by weight of the units derived from ethylene; and less than 20 percent by weight of units derived from one or more α-olefin comonomers; wherein the polyethylene composition has a density in the range of from 0.945 to 0.965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition.

The core of the bicomponent fiber comprises the first component. The first component comprises a polymeric material selected from the group consisting of polypropylene, polyester, polyamide, and combinations thereof. Polypropylene maybe a propylene homopolymer, a propylene copolymer such as a propylene alpha olefin copolymer, a random copolymer polypropylene.

The term (co)polymerization, as used herein, refers to the polymerization of ethylene and optionally one or more comonomers, e.g. one or more α-olefin comonomers. Thus, the term (co)polymerization refers to both polymerization of ethylene and copolymerization of ethylene and one or more comonomers, e.g. one or more α-olefin comonomers.

The sheath of the bicomponent fiber comprises the second component. The second component comprises a polyethylene composition as described below.

The polyethylene composition according to instant invention has a density in the range of 0.920 to 0.970 g/cm³. All individual values and subranges from 0.920 to 0.970 g/cm³ are included herein and disclosed herein; for example, the density can be from a lower limit of 0.920, 0.923, 0.928, 0.930, 0.936, 0.940, 0.945, 0.950, 0.955, or 0.960 g/cm³ to an upper limit of 0.941, 0.947, 0.954, 0.955, 0.959, 0.960, 0.965, 0.968, or 0.970 g/cm³. For example, the polyethylene composition may have a density in the range of 0.945 to 0.965 g/cm³; or in the alternative, the polyethylene composition may have a density in the range of 0.945 to 0.960 g/cm³; or in the alternative, the polyethylene composition may have a density in the range of 0.945 to 0.955 g/cm³; or in the alternative, the polyethylene composition may have a density in the range of 0.945 to 0.950 g/cm³; or in the alternative, the polyethylene composition may have a density in the range of 0.950 to 0.965 g/cm³; or in the alternative, the polyethylene composition may have a density in the range of 0.950 to 0.960 g/cm³; or in the alternative, the polyethylene composition may have a density in the range of 0.950 to 0.955 g/cm³.

The polyethylene composition according to the instant invention has a molecular weight distribution (M_{w}/Mₙ) in the range of 1.70 to 3.62. All individual values and subranges from 1.70 to 3.62 are included herein and disclosed herein; for example, the molecular weight distribution (M_{w}/Mₙ) can be from a lower limit of 1.70, 1.80, 1.90, 2.10, 2.30, 2.50, 2.70, 2.90, 3.10, 3.30, or 3.50 to an upper limit of 1.85, 1.95, 2.15, 2.35, 2.55, 2.75, 2.95, 3.15, 3.35, 3.50, 3.55, 3.60, or 3.62. For example, the polyethylene composition may have a molecular weight distribution (M_{w}/Mₙ) in the range of 1.70 to 3.50; or in the alternative, the polyethylene composition may have a molecular weight distribution (M_{w}/Mₙ) in the range of 1.70 to 3.49; or in the alternative, the polyethylene composition may have a molecular weight distribution (M_{w}/Mₙ) in the range of 1.70 to 3.45; or in the alternative, the polyethylene composition may have a molecular weight distribution (M_{w}/Mₙ) in the range of 1.70 to 3.35; or in the alternative, the polyethylene composition may have a molecular weight distribution (M_{w}/Mₙ) in the range of 1.70 to 3.15; or in the alternative, the polyethylene composition may have a molecular weight distribution (M_{w}/Mₙ) in the range of 1.70 to 2.95; or in the alternative, the polyethylene composition may have a molecular weight distribution (M_{w}/Mₙ) in the range of 1.70 to 2.75; or in the alternative, the polyethylene composition may have a molecular weight distribution (M_{w}/Mₙ) in the range of 1.70 to 2.55; or in the alternative, the polyethylene composition may have a molecular weight distribution (M_{w}/Mₙ) in the range of 1.70 to 2.35; or in the alternative, the polyethylene composition may have a molecular weight distribution (M_{w}/Mₙ) in the range of 1.70 to 2.15; or in the alternative, the polyethylene composition may have a molecular weight distribution (M_{w}/Mₙ) in the range of 1.70 to 1.95; or in the alternative, the polyethylene composition may have a molecular weight distribution (M_{w}/Mₙ) in the range of 1.70 to 1.85.

The polyethylene composition according to the instant invention has a melt index (I₂) in the range of 0.1 to 1000 g/10 minutes. All individual values and subranges from 0.1 to 1000 g/10 minutes are included herein and disclosed herein; for example, the melt index (I₂) can be from a lower limit of 0.1, 0.2, 0.5, 1, 2, 3, 5, 10, 20, 30, 40, 50, 60, 70, 80, or 100 g/10 minutes, to an upper limit of 5, 10, 30, 35, 50, 70, 80, 90, 100, 110, 150, 200, 220, 250, 300, 500, 800, or 1000 g/10 minutes. For example, the polyethylene composition may have a melt index (I₂) in the range of 0.2 to 150 g/10 minutes; or in the alternative, the polyethylene composition may have a melt index (I₂) in the range of 1 to 150 g/10 minutes; or in the alternative, the polyethylene composition may have a melt index (I₂) in the range of 10 to 150 g/10 minutes. The required polyethylene composition provides improved mechanical properties at low viscosities, which would allow higher throughput using bicomponent fiber technology, and thus, providing improved bicomponent fiber spinning process.

The polyethylene composition according to the instant invention has a molecular weight (M_{w}) in the range of 15,000 to 150,000 daltons. All individual values and subranges from 15,000 to 150,000 daltons are included herein and disclosed herein; for example, the molecular weight (M_{w}) can be from a lower limit of 15,000, 20,000, 25,000, 30,000, 34,000, 40,000, 50,000, 60,000, 70,000, 80,000, 90,000, 95,000, or 100,000 daltons to an upper limit of 20,000, 25,000, 30,000, 33,000, 40,000, 50,000, 60,000, 70,000, 80,000, 90,000, 95,000, 100,000, 115,000, 125,000, or 150,000. For example, the polyethylene composition may have a molecular weight (M_{w}) in the range of 15,000 to 125,000 daltons; or in the alternative, the polyethylene composition may have a molecular weight (M_{w}) in the range of 15,000 to 115,000 daltons; or in the alternative, the polyethylene composition may have a molecular weight (M_{w}) in the range of 15,000 to 100,000 daltons; or in the alternative, the polyethylene composition may have a molecular weight (M_{w}) in the range of 20,000 to 150,000 daltons; or in the alternative, the polyethylene composition may have a molecular weight (M_{w}) in the range of 30,000 to 150,000 daltons; or in the alternative, the polyethylene composition may have a molecular weight (M_{w}) in the range of 40,000 to 150,000 daltons; or in the alternative, the polyethylene composition may have a molecular weight (M_{w}) in the range of 50,000 to 150,000 daltons; or in the alternative, the polyethylene composition may have a molecular weight (M_{w}) in the range of 60,000 to 150,000 daltons; or in the alternative, the polyethylene composition may have a molecular weight (M_{w}) in the range of 80,000 to 150,000 daltons.

The polyethylene composition may have molecular weight distribution (M_{z}/M_{w}) in the range of less than 5. All individual values and subranges from less than 5 are included herein and disclosed herein; for example, the polyethylene composition may have a molecular weight distribution (M_{z}/M_{w}) in the range of less than 4.5; or in the alternative, the polyethylene composition may have a molecular weight distribution (M_{z}/M_{w}) in the range of less than 4; or in the alternative, the polyethylene composition may have a molecular weight distribution (M_{z}/M_{w}) in the range of less than 3.5; or in the alternative, the polyethylene composition may have a molecular weight distribution (M_{z}/M_{w}) in the range of less than 3.0; or in the alternative, the polyethylene composition may have a molecular weight distribution (M_{z}/M_{w}) in the range of less than 2.8; or in the alternative, the polyethylene composition may have a molecular weight distribution (M_{z}/M_{w}) in the range of less than 2.6; or in the alternative, the polyethylene composition may have a molecular weight distribution (M_{z}/M_{w}) in the range of less than 2.5; or in the alternative, the polyethylene composition may have a molecular weight distribution (M_{z}/M_{w}) in the range of less than 2.4; or in the alternative, the polyethylene composition may have a molecular weight distribution (M_{z}/M_{w}) in the range of less than 2.3; or in the alternative, the polyethylene composition may have a molecular weight distribution (M_{z}/M_{w}) in the range of less than 2.2.

The polyethylene composition may have a vinyl unsaturation of less than 0.1 vinyls per one thousand carbon atoms present in the backbone of the polyethylene composition. All individual values and subranges from less than 0.1 are included herein and disclosed herein; for example, the polyethylene composition may have a vinyl unsaturation of less than 0.08 vinyls per one thousand carbon atoms present in the backbone of the polyethylene composition; or in the alternative, the polyethylene composition may have a vinyl unsaturation of less than 0.06 vinyls per one thousand carbon atoms present in the backbone of the polyethylene composition; or in the alternative, the polyethylene composition may have a vinyl unsaturation of less than 0.04 vinyls per one thousand carbon atoms present in the backbone of the polyethylene composition; or in the alternative, the polyethylene composition may have a vinyl unsaturation of less than 0.02 vinyls per one thousand carbon atoms present in the backbone of the polyethylene composition; or in the alternative, the polyethylene composition may have a vinyl unsaturation of less than 0.01 vinyls per one thousand carbon atoms present in the backbone of the polyethylene composition; or in the alternative, the polyethylene composition may have a vinyl unsaturation of less than 0.001 vinyls per one thousand carbon atoms present in the backbone of the polyethylene composition.

The polyethylene composition may comprise less than 25 percent by weight of units derived from one or more α-olefin comonomers. All individual values and subranges from less than 25 weight percent are included herein and disclosed herein; for example, the polyethylene composition may comprise less than 20 percent by weight of units derived from one or more α-olefin comonomers; or in the alternative, the polyethylene composition may comprise less than 15 percent by weight of units derived from one or more α-olefin comonomers; or in the alternative, the polyethylene composition may comprise less than 12 percent by weight of units derived from one or more α-olefin comonomers; or in the alternative, the polyethylene composition may comprise less than 11 percent by weight of units derived from one or more α-olefin comonomers; or in the alternative, the polyethylene composition may comprise less than 9 percent by weight of units derived from one or more α-olefin comonomers; or in the alternative, the polyethylene composition may comprise less than 7 percent by weight of units derived from one or more α-olefin comonomers; or in the alternative, the polyethylene composition may comprise less than 5 percent by weight of units derived from one or more α-olefin comonomers; or in the alternative, the polyethylene composition may comprise less than 3 percent by weight of units derived from one or more α-olefin comonomers; or in the alternative, the polyethylene composition may comprise less than 1 percent by weight of units derived from one or more α-olefin comonomers; or in the alternative, the polyethylene composition may comprise less than 0.5 percent by weight of units derived from one or more α-olefin comonomers.

The α-olefin comonomers typically have no more than 20 carbon atoms. For example, the α-olefin comonomers may preferably have 3 to 10 carbon atoms, and more preferably 3 to 8 carbon atoms. Exemplary α-olefin comonomers include, but are not limited to, propylene, 1-butene, 1-pentene, 1-hexene, 1-heptene, 1-octene, 1-nonene, 1-decene, and 4-methyl-1-pentene. The one or more α-olefin comonomers may, for example, be selected from the group consisting of propylene, 1-butene, 1-hexene, and 1-octene; or in the alternative, from the group consisting of 1-hexene and 1-octene.

The polyethylene composition may comprise at least 75 percent by weight of units derived from ethylene. All individual values and subranges from at least 75 weight percent are included herein and disclosed herein; the polyethylene composition may comprise at least 80 percent by weight of units derived from ethylene; or in the alternative, for example, the polyethylene composition may comprise at least 85 percent by weight of units derived from ethylene; or in the alternative, the polyethylene composition may comprise at least 88 percent by weight of units derived from ethylene; or in the alternative, the polyethylene composition may comprise at least 89 percent by weight of units derived from ethylene; or in the alternative, the polyethylene composition may comprise at least 91 percent by weight of units derived from ethylene; or in the alternative, the polyethylene composition may comprise at least 93 percent by weight of units derived from ethylene; or in the alternative, the polyethylene composition may comprise at least 95 percent by weight of units derived from ethylene; or in the alternative, the polyethylene composition may comprise at least 97 percent by weight of units derived from ethylene; or in the alternative, the polyethylene composition may comprise at least 99 percent by weight of units derived from ethylene; or in the alternative, the polyethylene composition may comprise at least 99.5 percent by weight of units derived from ethylene.

The polyethylene composition of the instant invention is substantially free of any long chain branching, and preferably, the polyethylene composition of the instant invention is free of any long chain branching. Substantially free of any long chain branching, as used herein, refers to a polyethylene composition preferably substituted with less than about 0.1 long chain branching per 1000 total carbons, and more preferably, less than about 0.01 long chain branching per 1000 total carbons. In the alternative, the polyethylene composition of the instant invention is free of any long chain branching.

The polyethylene composition may have a short chain branching distribution breadth (SCBDB) in the range of 2 to 40° C. All individual values and subranges from 2 to 40° C. are included herein and disclosed herein; for example, the short chain branching distribution breadth (SCBDB) can be from a lower limit of 2, 3, 4, 5, 6, 8, 10, 12, 15, 18, 20, 25, or 30° C. to an upper limit of 40, 35, 30, 29, 27, 25, 22, 20, 15, 12, 10, 8, 6, 4, or 3° C. For example, the polyethylene composition may have a short chain branching distribution breadth (SCBDB) in the range of 2 to 35° C.; or in the alternative, the polyethylene composition may have a short chain branching distribution breadth (SCBDB) in the range of 2 to 30° C.; or in the alternative, the polyethylene composition may have a short chain branching distribution breadth (SCBDB) in the range of 2 to 25° C.; or in the alternative, the polyethylene composition may have a short chain branching distribution breadth (SCBDB) in the range of 2 to 20° C.; or in the alternative, the polyethylene composition may have a short chain branching distribution breadth (SCBDB) in the range of 2 to 15° C.; or in the alternative, the polyethylene composition may have a short chain branching distribution breadth (SCBDB) in the range of 2 to 10° C.; or in the alternative, the polyethylene composition may have a short chain branching distribution breadth (SCBDB) in the range of 2 to 5° C.; or in the alternative, the polyethylene composition may have a short chain branching distribution breadth (SCBDB) in the range of 4 to 35° C.; or in the alternative, the polyethylene composition may have a short chain branching distribution breadth (SCBDB) in the range of 4 to 30° C.; or in the alternative, the polyethylene composition may have a short chain branching distribution breadth (SCBDB) in the range of 4 to 25° C.; or in the alternative, the polyethylene composition may have a short chain branching distribution breadth (SCBDB) in the range of 4 to 20° C.; or in the alternative, the polyethylene composition may have a short chain branching distribution breadth (SCBDB) in the range of 4 to 15° C.; or in the alternative, the polyethylene composition may have a short chain branching distribution breadth (SCBDB) in the range of 4 to 10° C.; or in the alternative, the polyethylene composition may have a short chain branching distribution breadth (SCBDB) in the range of 4 to 5° C.

The inventive polyethylene composition may have a shear viscosity in the range of 20 to 250 Pascal-s at 3000 s⁻¹ shear rate measured at 190° C. All individual values and subranges from 20 to 250 Pascal-s at 3000 s⁻¹ shear rate measured at 190° C. are included herein and disclosed herein; for example, the polyethylene composition may have a shear viscosity in the range of 20 to 200 Pascal-s at 3000 s⁻¹ shear rate measured at 190° C.; or in the alternative, the polyethylene composition may have a shear viscosity in the range of 20 to 150 Pascal-s at 3000 s⁻¹ shear rate measured at 190° C.; or in the alternative, the polyethylene composition may have a shear viscosity in the range of 20 to 130 Pascal-s at 3000 s⁻¹ shear rate measured at 190° C.; or in the alternative, the polyethylene composition may have a shear viscosity in the range of 25 to 150 Pascal-s at 3000 s⁻¹ shear rate measured at 190° C.; or in the alternative, the polyethylene composition may have a shear viscosity in the range of 25 to 80 Pascal-s at 3000 s⁻¹ shear rate measured at 190° C.; or in the alternative, the polyethylene composition may have a shear viscosity in the range of 25 to 55 Pascal-s at 3000 s⁻¹ shear rate measured at 190° C.; or in the alternative, the polyethylene composition may have a shear viscosity in the range of 25 to 50 Pascal-s at 3000 s⁻¹ shear rate measured at 190° C.; or in the alternative, the polyethylene composition may have a shear viscosity in the range of 25 to 45 Pascal-s at 3000 s⁻¹ shear rate measured at 190° C.; or in the alternative, the polyethylene composition may have a shear viscosity in the range of 25 to 45 Pascal-s at 3000 s⁻¹ shear rate measured at 190° C.; or in the alternative, the polyethylene composition may have a shear viscosity in the range of 25 to 35 Pascal-s at 3000 s⁻¹ shear rate measured at 190° C.; or in the alternative, the polyethylene composition may have a shear viscosity in the range of 25 to 30 Pascal-s at 3000 s⁻¹ shear rate measured at 190° C.; or in the alternative, the polyethylene composition may have a shear viscosity in the range of 30 to 55 Pascal-s at 3000 s⁻¹ shear rate measured at 190° C.; or in the alternative, the polyethylene composition may have a shear viscosity in the range of 35 to 55 Pascal-s at 3000 s⁻¹ shear rate measured at 190° C.; or in the alternative, the polyethylene composition may have a shear viscosity in the range of 40 to 55 Pascal-s at 3000 s⁻¹ shear rate measured at 190° C.; or in the alternative, the polyethylene composition may have a shear viscosity in the range of 45 to 55 Pascal-s at 3000 s⁻¹ shear rate measured at 190° C.; or in the alternative, the polyethylene composition may have a shear viscosity in the range of 50 to 55 Pascal-s at 3000 s⁻¹ shear rate measured at 190° C.

The inventive polyethylene composition may further comprise less than or equal to 100 parts by weight of hafnium residues remaining from the hafnium based metallocene catalyst per one million parts of polyethylene composition. All individual values and subranges from less than or equal to 100 ppm are included herein and disclosed herein; for example, the polyethylene composition may further comprise less than or equal to 10 parts by weight of hafnium residues remaining from the hafnium based metallocene catalyst per one million parts of polyethylene composition; or in the alternative, the polyethylene composition may further comprise less than or equal to 8 parts by weight of hafnium residues remaining from the hafnium based metallocene catalyst per one million parts of polyethylene composition; or in the alternative, the polyethylene composition may further comprise less than or equal to 6 parts by weight of hafnium residues remaining from the hafnium based metallocene catalyst per one million parts of polyethylene composition; or in the alternative, the polyethylene composition may further comprise less than or equal to 4 parts by weight of hafnium residues remaining from the hafnium based metallocene catalyst per one million parts of polyethylene composition; or in the alternative, the polyethylene composition may further comprise less than or equal to 2 parts by weight of hafnium residues remaining from the hafnium based metallocene catalyst per one million parts of polyethylene composition; or in the alternative, the polyethylene composition may further comprise less than or equal to 1.5 parts by weight of hafnium residues remaining from the hafnium based metallocene catalyst per one million parts of polyethylene composition; or in the alternative, the polyethylene composition may further comprise less than or equal to 1 parts by weight of hafnium residues remaining from the hafnium based metallocene catalyst per one million parts of polyethylene composition; or in the alternative, the polyethylene composition may further comprise less than or equal to 0.75 parts by weight of hafnium residues remaining from the hafnium based metallocene catalyst per one million parts of polyethylene composition; or in the alternative, the polyethylene composition may further comprise less than or equal to 0.5 parts by weight of hafnium residues remaining from the hafnium based metallocene catalyst per one million parts of polyethylene composition the polyethylene composition may further comprise from 0.1 to 100 parts by weight of hafnium residues remaining from the hafnium based metallocene catalyst per one million parts of polyethylene composition. The hafnium residues remaining from the hafnium based metallocene catalyst in the inventive polyethylene composition may be measured by x-ray fluorescence (XRF), which is calibrated to reference standards. The polymer resin granules were compression molded at elevated temperature into plaques having a thickness of about ⅜ of an inch for the x-ray measurement in a preferred method. At very low concentrations of metal, such as below 0.1 ppm, ICP-AES would be a suitable method to determine metal residues present in the inventive polyethylene composition. In one embodiment, the inventive polyethylene composition has substantially no chromium, zirconium or titanium content, that is, no or only what would be considered by those skilled in the art, trace amounts of these metals are present, such as, for example, less than 0.001 ppm.

The inventive polyethylene composition in accordance with the instant invention may have less than 2 peaks on an elution temperature-eluted amount curve determined by continuous temperature rising elution fraction method at equal or above 30° C., wherein the purge peak which is below 30° C. is excluded. In the alternative, the polyethylene composition may have only 1 peak or less on an elution temperature-eluted amount curve determined by continuous temperature rising elution fraction method at equal or above 30° C., wherein the purge peak which is below 30° C. is excluded. In the alternative, the polyethylene composition may have only 1 peak on an elution temperature-eluted amount curve determined by continuous temperature rising elution fraction method at equal or above 30° C., wherein the purge peak which is below 30° C. is excluded. In addition, artifacts generated due to instrumental noise at either side of a peak are not considered to be peaks.

The inventive polyethylene composition may further comprise additional components such as one or more other polymers and/or one or more additives. Such additives include, but are not limited to, antistatic agents, color enhancers, dyes, lubricants, fillers, pigments, primary antioxidants, secondary antioxidants, processing aids, UV stabilizers, anti-blocks, slip agents, tackifiers, fire retardants, anti-microbial agents, odor reducer agents, anti fungal agents, and combinations thereof. The inventive polyethylene composition may contain any amounts of additives. The inventive polyethylene composition may comprise from about 0.1 to about 10 percent by the combined weight of such additives, based on the weight of the inventive polyethylene composition including such additives. All individual values and subranges from about 0.1 to about 10 weight percent are included herein and disclosed herein; for example, the inventive polyethylene composition may comprise from 0.1 to 7 percent by the combined weight of additives, based on the weight of the inventive polyethylene composition including such additives; in the alternative, the inventive polyethylene composition may comprise from 0.1 to 5 percent by the combined weight of additives, based on the weight of the inventive polyethylene composition including such additives; or in the alternative, the inventive polyethylene composition may comprise from 0.1 to 3 percent by the combined weight of additives, based on the weight of the inventive polyethylene composition including such additives; or in the alternative, the inventive polyethylene composition may comprise from 0.1 to 2 percent by the combined weight of additives, based on the weight of the inventive polyethylene composition including such additives; or in the alternative, the inventive polyethylene composition may comprise from 0.1 to 1 percent by the combined weight of additives, based on the weight of the inventive polyethylene composition including such additives; or in the alternative, the inventive polyethylene composition may comprise from 0.1 to 0.5 percent by the combined weight of additives, based on the weight of the inventive polyethylene composition including such additives. Antioxidants, such as Irgafos™ 168, Irganox™ 3114, Cyanox™ 1790, Irganox™ 1010, Irganox™ 1076, Irganox™1330, Irganox™1425WL, Irgastab™ may be used to protect the inventive polyethylene composition from thermal and/or oxidative degradation. Irganox™ 1010 is tetrakis (methylene (3,5-di-tert-butyl-4hydroxyhydrocinnamate), commercially available from Ciba Geigy Inc.; Irgafos™ 168 is tris (2,4 di-tert-butylphenyl)phosphite, commercially available from Ciba Geigy Inc.; Irganox™ 3114 is [1,3,5-Tris(3,5-di-(tert)-butyl-4-hydroxybenzyl)-1,3,5-triazine-2,4,6(1H,3H,5H)-trione], commercially available from Ciba Geigy Inc.; Irganox™ 1076 is (Octadecyl 3,5-di-tert-butyl-4 hydroxycinnamate), commercially available from Ciba Geigy Inc.; Irganox™1330 is [1,3,5-Trimethyl-2,4,6-tris(3,5-di-tert-butyl-4-hydroxybenzyl)benzene], commercially available from Ciba Geigy Inc.; Irganox™1425WL is (Calcium bis[fluoriding(3,5-di-(tert)-butyl-4-hydroxybenzyl)phosphonate]), commercially available from Ciba Geigy Inc.; Irgastab™is [bis(hydrogenated tallow alkyl)amines, oxidized], commercially available from Ciba Geigy Inc.; Cyanox™ 1790is [Tris (4-t-butyl-3-hydroxy-2,6-dimethylbenzyl)-s-triazine-2,4,6-(1H,3H,5H)-trione], commercially available from Cytec Industries, Inc. Other commercially available antioxidants include, but are not limited to, Ultranox™ 626, a Bis (2,4-di-t-butylphenyl)Pentaerythritol Diphosphite, commercially available from Chemtura Corporation; P-EPQ™, a Phosphonous acid, P,P′-[[1,1′-biphenyl]-4,4′-diyl]bis-, P,P,P′,P′-tetrakis[2,4-bis(1,1-dimethylethyl)phenyl]ester, commercially available from Clariant Corporation; Doverphos™ 9228, a Bis (2,4-decumylphenyl)Pentaerythritol Diphosphite, commercially available from Dover Chemical Corporation; Chimassorb™ 944, a Poly[[6-[(1,1,3,3-tetramethylbutyl)amino]-1,3,5-triazine-2,4-diyl][(2,2,6,6-tetramethyl-4-piperidinyl)imino]-1,6-hexanediyl[(2,2,6,6-tetramethyl-4-piperidinyl)imino]], commercially available from Ciba Geigy Inc.; Chimassorb™ 119, a 1,3,5-Triazine-2,4,6-triamine, N2,N2′-1,2-ethanediylbis[N2-[3-[[4,6-bis[butyl(1,2,2,6,6-pentamethyl-4-piperidinyl)amino]-1,3,5-triazin-2-yl]amino]propyl]-N4,N6-dibutyl-N4,N6-bis(1,2,2,6,6-pentamethyl-4-piperidinyl)-, commercially available from Ciba Geigy Inc.; Chimassorb™ 2020, a Poly[[6-[butyl(2,2,6,6-tetramethyl-4-piperidinyl)amino]-1,3,5-triazine-2,4-diyl][(2,2,6,6-tetramethyl-4-piperidinyl)imino]-1,6-hexanediyl[(2,2,6,6-tetramethyl-4-piperidinyl)amino], α- [[6-[[4,6-bis(dibutylamino)-1,3,5-triazin-2-yl](2,2,6,6-tetramethyl-4-piperidinyl)amino]hexyl](2,2,6,6-tetramethyl-4-piperidinyl)amino]-ω-[4,6-bis(dibutylamino)-1,3,5-triazin-2-yl]-, commercially available from Ciba Geigy Inc.; Tinuvin™ 622, a Butanedioic acid polymer with 4-hydroxy-2,2,6,6-tetramethyl-1-piperidineethanol, commercially available from Ciba Geigy Inc.; Tinuvin™ 770, a Decanedioic acid, 1,10-bis(2,2,6,6-tetramethyl-4-piperidinyl)ester, commercially available from Ciba Geigy Inc.; Uvasorb HA™ 88, a 2,5-Pyrrolidinedione, 3-dodecyl-1-(2,2,6,6-tetramethyl-4-piperidinyl), commercially available from 3V; CYASORB™ UV-3346, a Poly[[6-(4-morpholinyl)-1,3,5-triazine-2,4-diyl][(2,2,6,6-tetramethyl-4-piperidinyl)imino]-1,6-hexanediyl[(2,2,6,6-tetramethyl-4-piperidinyl)imino]], commercially available from Cytec Industries, Inc.; CYASORB™ UV-3529, a Poly[[6-(4-morpholinyl)-1,3,5-triazine-2,4-diyl][(1,2,2,6,6-pentamethyl-4-piperidinyl)imino]-1,6-hexanediyl[(1,2,2,6,6-pentamethyl-4-piperidinyl)imino]], commercially available from Cytec Industries, Inc.; and Hostavin™ N 30, a 7-Oxa-3,20-iazadispiro[5.1.11.2]heneicosan-21-one, 2,2,4,4-tetramethyl-20-(2-oxiranylmethyl)-, polymer with 2-(chloromethyl)oxirane, commercially available from Clariant Corporation.

Any conventional ethylene (co)polymerization reaction processes may be employed to produce the inventive polyethylene composition. Such conventional ethylene (co)polymerization reaction processes include, but are not limited to, gas phase polymerization process, slurry phase polymerization process, liquid phase polymerization process, and combinations thereof using one or more conventional reactors, e.g. fluidized bed gas phase reactors, loop reactors, stirred tank reactors, batch reactors in parallel, series, and/or any combinations thereof. In the alternative, the inventive polyethylene composition may be produced in a high pressure reactor via a coordination catalyst system. For example, the inventive polyethylene composition may be produced via gas phase polymerization process in a single gas phase reactor; however, the instant invention is not so limited, and any of the above polymerization processes may be employed. In one embodiment, the polymerization reactor may comprise of two or more reactors in series, parallel, or combinations thereof. Preferably, the polymerization reactor is a single reactor, e.g. a fluidized bed gas phase reactor. In another embodiment, the gas phase polymerization reactor is a continuous polymerization reactor comprising one or more feed streams. In the polymerization reactor, the one or more feed streams are combined together, and the gas comprising ethylene and optionally one or more comonomers, e.g. one or more α-olefins, are flowed or cycled continuously through the polymerization reactor by any suitable means. The gas comprising ethylene and optionally one or more comonomers, e.g. one or more α-olefins, may be fed up through a distributor plate to fluidize the bed in a continuous fluidization process.

In production, a hafnium based metallocene catalyst system including a cocatalyst, as described hereinbelow in further details, ethylene, optionally one or more alpha-olefin comonomers, hydrogen, optionally one or more inert gases and/or liquids, e.g. N₂, isopentane, and hexane, and optionally one or more continuity additive, e.g. ethoxylated stearyl amine or aluminum distearate or combinations thereof, are continuously fed into a reactor, e.g. a fluidized bed gas phase reactor. The reactor may be in fluid communication with one or more discharge tanks, surge tanks, purge tanks, and/or recycle compressors. The temperature in the reactor is typically in the range of 70 to 115° C., preferably 75 to 110° C., more preferably 75 to 100° C., and the pressure is in the range of 15 to 30 atm, preferably 17 to 26 atm. A distributor plate at the bottom of the polymer bed provides a uniform flow of the upflowing monomer, comonomer, and inert gases stream. A mechanical agitator may also be provided to facilitate contact between the solid particles and the comonomer gas stream. The fluidized bed, a vertical cylindrical reactor, may have a bulb shape at the top to facilitate the reduction of gas velocity; thus, permitting the granular polymer to separate from the upflowing gases. The unreacted gases are then cooled to remove the heat of polymerization, recompressed, and then recycled to the bottom of the reactor. Once resin is removed from the reactor, it is transported to a purge bin to purge the residual hydrocarbons. Moisture may be introduced to react with residual catalyst and co-catalyst prior to exposure and reaction with oxygen. The inventive polyethylene composition may then be transferred to an extruder to be pelletized. Such pelletization techniques are generally known. The inventive polyethylene composition may further be melt screened. Subsequent to the melting process in the extruder, the molten composition is passed through one or more active screens, positioned in series of more than one, with each active screen having a micron retention size of from about 2 μm to about 400 μm (2 to 4×10⁻⁵ m), and preferably about 2 μm to about 300 μm (2 to 3×10⁻⁵ m), and most preferably about 2 μm to about 70 μm (2 to 7×10⁻⁶ m), at a mass flux of about 5 to about 100 lb/hr/in² (1.0 to about 20 kg/s/m²). Such further melt screening is disclosed in U.S. Pat. No. 6,485,662, which is incorporated herein by reference to the extent that it discloses melt screening.

In an embodiment of a fluidized bed reactor, a monomer stream is passed to a polymerization section. The fluidized bed reactor may include a reaction zone in fluid communication with a velocity reduction zone. The reaction zone includes a bed of growing polymer particles, formed polymer particles and catalyst composition particles fluidized by the continuous flow of polymerizable and modifying gaseous components in the form of make-up feed and recycle fluid through the reaction zone. Preferably, the make-up feed includes polymerizable monomer, most preferably ethylene and optionally one or more α-olefin comonomers, and may also include condensing agents as is known in the art and disclosed in, for example, U.S. Pat. No. 4,543,399, U.S. Pat. No. 5,405,922, and U.S. Pat. No. 5,462,999.

The fluidized bed has the general appearance of a dense mass of individually moving particles, preferably polyethylene particles, as created by the percolation of gas through the bed. The pressure drop through the bed is equal to or slightly greater than the weight of the bed divided by the cross-sectional area. It is thus dependent on the geometry of the reactor. To maintain a viable fluidized bed in the reaction zone, the superficial gas velocity through the bed must exceed the minimum flow required for fluidization. Preferably, the superficial gas velocity is at least two times the minimum flow velocity. Ordinarily, the superficial gas velocity does not exceed 1.5 m/sec and usually no more than 0.76 m/sec is sufficient.

In general, the height to diameter ratio of the reaction zone can vary in the range of about 2:1 to about 5:1. The range, of course, can vary to larger or smaller ratios and depends upon the desired production capacity. The cross-sectional area of the velocity reduction zone is typically within the range of about 2 to about 3 multiplied by the cross-sectional area of the reaction zone.

The velocity reduction zone has a larger inner diameter than the reaction zone, and can be conically tapered in shape. As the name suggests, the velocity reduction zone slows the velocity of the gas due to the increased cross sectional area. This reduction in gas velocity drops the entrained particles into the bed, reducing the quantity of entrained particles that flow from the reactor. The gas exiting the overhead of the reactor is the recycle gas stream.

The recycle stream is compressed in a compressor and then passed through a heat exchange zone where heat is removed before the stream is returned to the bed. The heat exchange zone is typically a heat exchanger, which can be of the horizontal or vertical type. If desired, several heat exchangers can be employed to lower the temperature of the cycle gas stream in stages. It is also possible to locate the compressor downstream from the heat exchanger or at an intermediate point between several heat exchangers. After cooling, the recycle stream is returned to the reactor through a recycle inlet line. The cooled recycle stream absorbs the heat of reaction generated by the polymerization reaction.

Preferably, the recycle stream is returned to the reactor and to the fluidized bed through a gas distributor plate. A gas deflector is preferably installed at the inlet to the reactor to prevent contained polymer particles from settling out and agglomerating into a solid mass and to prevent liquid accumulation at the bottom of the reactor as well to facilitate easy transitions between processes that contain liquid in the cycle gas stream and those that do not and vice versa. Such deflectors are described in the U.S. Pat. No. 4,933,149 and U.S. Pat. No. 6,627,713.

The hafnium based catalyst system used in the fluidized bed is preferably stored for service in a reservoir under a blanket of a gas, which is inert to the stored material, such as nitrogen or argon. The hafnium based catalyst system is injected into the bed at a point above distributor plate. Preferably, the hafnium based catalyst system is injected at a point in the bed where good mixing with polymer particles occurs. Injecting the hafnium based catalyst system at a point above the distribution plate facilitates the operation of a fluidized bed polymerization reactor.

The monomers can be introduced into the polymerization zone in various ways including, but not limited to, direct injection through a nozzle into the bed or cycle gas line. The monomers can also be sprayed onto the top of the bed through a nozzle positioned above the bed, which may aid in eliminating some carryover of fines by the cycle gas stream.

Make-up fluid may be fed to the bed through a separate line to the reactor. A gas analyzer determines the composition of the recycle stream, and the composition of the make-up stream is adjusted accordingly to maintain an essentially steady state gaseous composition within the reaction zone. The gas analyzer can be a conventional gas analyzer that determines the recycle stream composition to maintain the ratios of feed stream components. Such equipment is commercially available from a wide variety of sources. The gas analyzer is typically positioned to receive gas from a sampling point located between the velocity reduction zone and heat exchanger.

The production rate of inventive polyethylene composition may be conveniently controlled by adjusting the rate of catalyst composition injection, monomer concentration, or both. Since any change in the rate of catalyst composition injection will change the reaction rate and thus the rate at which heat is generated in the bed, the temperature of the recycle stream entering the reactor is adjusted to accommodate any change in the rate of heat generation. This ensures the maintenance of an essentially constant temperature in the bed. Complete instrumentation of both the fluidized bed and the recycle stream cooling system is, of course, useful to detect any temperature change in the bed so as to enable either the operator or a conventional automatic control system to make a suitable adjustment in the temperature of the recycle stream.

Under a given set of operating conditions, the fluidized bed is maintained at essentially a constant height by withdrawing a portion of the bed as product at the rate of formation of the particulate polymer product. Since the rate of heat generation is directly related to the rate of product formation, a measurement of the temperature rise of the fluid across the reactor, i.e. the difference between inlet fluid temperature and exit fluid temperature, is indicative of the rate of inventive polyethylene composition formation at a constant fluid velocity if no or negligible vaporizable liquid is present in the inlet fluid.

On discharge of particulate polymer product from reactor, it is desirable and preferable to separate fluid from the product and to return the fluid to the recycle line. There are numerous ways known to the art to accomplish this separation. Product discharge systems which may be alternatively employed are, for example, disclosed and claimed in U.S. Pat. No. 4,621,952. Such a system typically employs at least one (parallel) pair of tanks comprising a settling tank and a transfer tank arranged in series and having the separated gas phase returned from the top of the settling tank to a point in the reactor near the top of the fluidized bed.

In the fluidized bed gas phase reactor embodiment, the reactor temperature of the fluidized bed process herein ranges from 70° C. or 75° C., or 80° C. to 90° C. or 95° C., or 100° C., or 110° C., or 115° C. , wherein a desirable temperature range comprises any upper temperature limit combined with any lower temperature limit described herein. In general, the reactor temperature is operated at the highest temperature that is feasible, taking into account the sintering temperature of the inventive polyethylene composition within the reactor and fouling that may occur in the reactor or recycle line(s) as well as the impact on the inventive polyethylene composition and catalyst productivity.

The process of the present invention is suitable for the production of homopolymers comprising ethylene derived units, or copolymers comprising ethylene derived units and at least one or more other α-olefin(s) derived units.

In order to maintain an adequate catalyst productivity in the present invention, it is preferable that the ethylene is present in the reactor at a partial pressure at or greater than 160 psia (1100 kPa), or 190 psia (1300 kPa), or 200 psia (1380 kPa), or 210 psia (1450 kPa), or 220 psia (1515 kPa), or 230 psia (1585 kPa), or 240 psia (1655 pKa).

The comonomer, e.g. one or more α-olefin comonomers, if present in the polymerization reactor, is present at any level that will achieve the desired weight percent incorporation of the comonomer into the finished polyethylene. This may be expressed as a mole ratio of comonomer to ethylene as described herein, which is the ratio of the gas concentration of comonomer moles in the cycle gas to the gas concentration of ethylene moles in the cycle gas. In one embodiment of the inventive polyethylene composition production, the comonomer is present with ethylene in the cycle gas in a mole ratio range of from 0 to 0.1 (comonomer:ethylene); and from 0 to 0.05 in another embodiment; and from 0 to 0.04 in another embodiment; and from 0 to 0.03 in another embodiment; and from 0 to 0.02 in another embodiment.

Hydrogen gas may also be added to the polymerization reactor(s) to control the final properties (e.g., I₂₁ and/or I₂) of the inventive polyethylene composition. In one embodiment, the ratio of hydrogen to total ethylene monomer (ppm H₂/mol % C₂) in the circulating gas stream is in a range of from 0 to 60:1; in another embodiment, from 0.10:1 (0.10) to 50:1 (50); in another embodiment, from 0 to 35:1 (35); in another embodiment, from 0 to 25:1 (25); in another embodiment, from 7:1 (7) to 22:1 (22).

The hafnium based catalyst system, as used herein, refers to a catalyst composition capable of catalyzing the polymerization of ethylene monomers and optionally one or more α-olefin co monomers to produce polyethylene. Furthermore, the hafnium based catalyst system comprises a hafnocene component. The hafnocene component may have an average particle size in the range of 12 to 35 μm; for example, the hafnocene component may have an average particle size in the range of 20 to 30 μm, e.g. 25μ. The hafnocene component may comprise mono-, bis- or tris-cyclopentadienyl-type complexes of hafnium. In one embodiment, the cyclopentadienyl-type ligand comprises cyclopentadienyl or ligands isolobal to cyclopentadienyl and substituted versions thereof. Representative examples of ligands isolobal to cyclopentadienyl include, but are not limited to, cyclopentaphenanthreneyl, indenyl, benzindenyl, fluorenyl, octahydrofluorenyl, cyclooctatetraenyl, cyclopentacyclododecene, phenanthrindenyl, 3,4-benzofluorenyl, 9-phenylfluorenyl, 8-H-cyclopent[a]acenaphthylenyl, 7H-dibenzofluorenyl, indeno[1,2-9]anthrene, thiophenoindenyl, thiophenofluorenyl, hydrogenated versions thereof (e.g., 4,5,6,7-tetrahydroindenyl, or “H₄Ind”) and substituted versions thereof. In one embodiment, the hafnocene component is an unbridged bis-cyclopentadienyl hafnocene and substituted versions thereof. In another embodiment, the hafnocene component excludes unsubstituted bridged and unbridged bis-cyclopentadienyl hafnocenes, and unsubstituted bridged and unbridged bis-indenyl hafnocenes. The term “unsubstituted,” as used herein, means that there are only hydride groups bound to the rings and no other group. Preferably, the hafnocene useful in the present invention can be represented by the formula (where “Hf” is hafnium):

CpₙHfXₚ (1)

wherein ₙ is 1 or 2, ₚ is 1, 2 or 3, each Cp is independently a cyclopentadienyl ligand or a ligand isolobal to cyclopentadienyl or a substituted version thereof bound to the hafnium; and X is selected from the group consisting of hydride, halides, C₁ to C₁₀ alkyls and C₂ to C₁₂ alkenyls; and wherein whenₙ is 2, each Cp may be bound to one another through a bridging group A selected from the group consisting of C₁ to C₅ alkylenes, oxygen, alkylamine, silyl-hydrocarbons, and siloxyl-hydrocarbons. An example of C₁ to C₅ alkylenes include ethylene (—CH₂CH₂—) bridge groups; an example of an alkylamine bridging group includes methylamide (—(CH₃)N—); an example of a silyl-hydrocarbon bridging group includes dimethylsilyl (—(CH₃)₂Si—); and an example of a siloxyl-hydrocarbon bridging group includes (—O(CH₃)₂Si—O—). In one particular embodiment, the hafnocene component is represented by formula (1), wherein ₙ is 2 and ₚ is 1 or 2.

As used herein, the term “substituted” means that the referenced group possesses at least one moiety in place of one or more hydrogens in any position, the moieties selected from such groups as halogen radicals such as F, Cl, Br, hydroxyl groups, carbonyl groups, carboxyl groups, amine groups, phosphine groups, alkoxy groups, phenyl groups, naphthyl groups, C₁ to C₁₀ alkyl groups, C₂ to C₁₀ alkenyl groups, and combinations thereof. Examples of substituted alkyls and aryls includes, but are not limited to, acyl radicals, alkylamino radicals, alkoxy radicals, aryloxy radicals, alkylthio radicals, dialkylamino radicals, alkoxycarbonyl radicals, aryloxycarbonyl radicals, carbamoyl radicals, alkyl- and dialkyl-carbamoyl radicals, acyloxy radicals, acylamino radicals, arylamino radicals, and combinations thereof. More preferably, the hafnocene component useful in the present invention can be represented by the formula:

(CpR₅)₂HfX₂ (2)

wherein each Cp is a cyclopentadienyl ligand and each is bound to the hafnium; each R is independently selected from hydrides and C₁ to C₁₀ alkyls, most preferably hydrides and C₁ to C₅ alkyls; and X is selected from the group consisting of hydride, halide, C₁ to C₁₀ alkyls and C₂ to C₁₂ alkenyls, and more preferably X is selected from the group consisting of halides, C₂ to C₆ alkylenes and C₁ to C₆ alkyls, and most preferably X is selected from the group consisting of chloride, fluoride, C₁ to C₅ alkyls and C₂ to C₆ alkylenes. In a most preferred embodiment, the hafnocene is represented by formula (2) above, wherein at least one R group is an alkyl as defined above, preferably a C₁ to C₅ alkyl, and the others are hydrides. In a most preferred embodiment, each Cp is independently substituted with from one, two, or three groups selected from the group consisting of methyl, ethyl, propyl, butyl, and isomers thereof.

In one embodiment, the hafnocene based catalyst system is heterogeneous, i.e. the hafnocene based catalyst may further comprise a support material. The support material can be any material known in the art for supporting catalyst compositions; for example, an inorganic oxide; or in the alternative, silica, alumina, silica-alumina, magnesium chloride, graphite, magnesia, titania, zirconia, and montmorillonite, any of which can be chemically/physically modified such as by fluoriding processes, calcining or other processes known in the art. In one embodiment the support material is a silica material having an average particle size as determined by Malvern analysis of from 1 to 60 mm; or in the alternative, 10 to 40 mm.

In one embodiment, the hafnocene component may be spray-dried hafnocene composition containing a micro-particulate filler such as Cabot TS-610.

The hafnocene based catalyst system may further comprise an activator. Any suitable activator known to activate catalyst components for olefin polymerization may be suitable. In one embodiment, the activator is an alumoxane; in the alternative methalumoxane such as described by J. B. P. Soares and A. E. Hamielec in 3(2) POLYMER REACTION ENGINEERING, 131-200 (1995). The alumoxane may preferably be co-supported on the support material in a molar ratio of aluminum to hafnium (Al:Hf) ranging from 80:1 to 200:1, most preferably 90:1 to 140:1.

Such hafnium based catalyst systems are further described in details in the U.S. Pat. No. 6,242,545 and U.S. Pat. No. 7,078,467, incorporated herein by reference.

The fibers according to the instant invention comprise the above polyethylene composition, and optionally one or more other polymers. The inventive fibers may have a denier per filament in the range of less than 50 g/9000 m. All individual values and subranges from less than 50 g/9000 m are included herein and disclosed herein; for example, the denier per filament can be from a lower limit of 0.1, 0.5, 1, 1.6, 1.8, 2.0. 2.2, 2.4, 5, 10, 15, 17, 20, 25, 30, 33, 40, or 44 g/9000 m to an upper limit of 0.5, 1, 1.7, 1.8, 1.9, 2.0, 2.1, 2.2, 2.3, 2.4, 5, 10, 15, 17, 20, 25, 30, 33, 40, 44, or 50 g/9000 m. For example, the inventive fibers may have a denier per filament in the range of less than 40 g/9000 m; or in the alternative, the inventive fibers may have a denier per filament in the range of from 0.1 to 10 g/9000 m; or in the alternative, the inventive fibers may have a denier per filament in the range of from 1 to 5 g/9000 m; or in the alternative, the inventive fibers may have a denier per filament in the range of from 0.1 to 5 g/9000 m; or in the alternative, the inventive fibers may have a denier per filament in the range of from 0.1 to 2.6 g/9000 m; or in the alternative, the inventive fibers may have a denier per filament in the range of from 1 to 3 g/9000 m; or in the alternative, the inventive fibers may have a denier per filament in the range of from 1 to 2.5 g/9000 m; or in the alternative, the inventive fibers may have a denier per filament in the range of from 1.5 to 3 g/9000 m; or in the alternative, the inventive fibers may have a denier per filament in the range of from 1.6 to 2.4 g/9000 m.

Inventive fibers according to the instant invention may be produced via different techniques. The inventive fibers may, for example, be produced via melt spinning. The inventive fibers according to instant invention may be continuous filaments, or in the alternative, the inventive fibers may be staple fibers. Continuous filaments may further be optionally crimped, and then cut to produce staple fibers. The inventive fibers include, but are not limited to, bi-component fibers, and/or multi-component fibers. Exemplary bi-component fibers include, but are not limited to, sheath/core, islands in the sea, segmented pie, and combination thereof. The inventive fibers may include the polyethylene composition according to the instant invention as an outer layer, e.g. sheath, alone or in combination with one or more polymers. The inventive fibers may include the inventive polyethylene composition according to the instant invention as an inner layer, e.g. core, alone or in combination with one or more polymers. The inventive fibers or the inventive fiber components, i.e. inner layer and outer layer, according to the instant invention may be mono-constituent, i.e. only inventive polyethylene composition; or in the alternative, the inventive fibers or the inventive fiber components, i.e. inner layer and outer layer according to the instant invention may be multi-constituent, i.e. a blend of inventive polyethylene composition and one or more polymers. The term outer layer, as used herein, refers to at least any portion of the fiber surface. The term inner layer, as used herein, refers to any portion below the fiber surface.

In melt spinning, the inventive polyethylene composition is melt extruded and forced through the fine orifices in a metallic plate called spinneret into air or other gas, where it is cooled and solidified. The solidified filaments may be drawn-off via rotating rolls, or godets, and wound onto bobbins.

Inventive fabrics according to instant invention include, but are not limited to, non-woven fabrics, woven fabrics, and combination thereof.

The non-woven fabrics according to the instant invention may be fabricated via different techniques. Such methods include, but are not limited to, melt blown process, spunbond process, carded web process, air laid process, thermo-calendering process, adhesive bonding process, hot air bonding process, needle punch process, hydroentangling process, electrospinning process, and combinations thereof.

In melt blown process, the inventive non-woven fabric is formed by extruding molten polyethylene composition of the instant invention through a die, then, attenuating and/or optionally breaking the resulting filaments with hot, high-velocity air or stream thereby forming short or long fiber lengths collected on a moving screen where they bond during cooling.

In the alternative, the melt blown process generally includes the following steps: (a) Extruding strands from a spinneret; (b) Simultaneously quenching and attenuating the polymer stream immediately below the spinneret using streams of high velocity heated air; (c) Collecting the drawn strands into a web on a foraminous surface. Meltblown webs can be bonded by a variety of means including, but not limited to, autogeneous bonding, i.e. self bonding without further treatment, thermo-calendering process, adhesive bonding process, hot air bonding process, needle punch process, hydroentangling process, and combinations thereof.

Spunbonded products are non-woven fabrics formed by filaments that have been extruded, drawn, but then laid on a continuous belt. Bonding is accomplished by several methods such as by hot roll calendaring or bypassing the web through a saturated steam chamber at an elevated pressure. Nonwoven fabric is an assembly of textile fibers held together by fusing of the fibers. Initially, the fibers may be oriented in one direction or may be deposited in a random matter. This web of fibers is then bonded together. This spunbond process is a nonwoven manufacturing system involving the direct conversion of a polymer into continuous filaments, integrated with the conversion of the filaments into a random laid, bonded nonwoven fabric. In general, the spunbond nonwoven process consists of several integrated steps in the conversion and a polymer into a finished nonwoven fabric. First, the polymer feedstock in pellet or powder form is conveyed from storage bins to the feeder section of an extruder. The polymer feed is mixed with stabilizers, additives, color master bath, resin modifiers, or other additives, and this blend of raw material is melted within the extruder barrel. The molten polymer mix is pumped through a heated conduit to a resin filter system, and into a distributor section that leads to the spinneret units. In spinneret usually consist of a perforated plate arranged across the width of the line. The resin is forced through the many small holes in the spinneret plate to form continuous filaments. As the filaments emerge through the spinneret holes, they are directed downward into quench chambers or chimneys. As the filaments travel through these chambers, cool air is directed across the filament bundle to cool the molten filaments sufficiently to cause solidification. The filaments are then led further downward into a tapered conduit by an air stream. A second stream of high velocity air is directed parallel to the direction of the filaments, causing an acceleration and accompanying attenuation were stretching of the individual filaments. This mechanical stretching results in increased orientation of the polymer chains making up the continuous filaments. Such orientation leads to increased filament strength, along with modification of the other filament properties, including the filament denier or thickness. The filaments are deposited in a random manner on a moving, porous forming belt. A vacuum under the belt assists in forming the filament web under forming belt, and in removing the air used in the extrusion and/or orientation operation. In some processes, an electrostatic charge is placed on the filament bundle to ensure spreading and separation of individual filaments. In other processes, deflector plates are used to lay down the filament sheet in a random manner on the forming belt. The continuous filament web is delivered to a bonding section, where one of several bonding methods can be used to bond the loose elements into a strong, integrated fabric. The bonded fabric may encounter a slitting section where the two edges are trimmed to eliminate nonuniform rough edge created during the manufacturing step. In some operations, the fabric may also be further slit into precise, smaller widths to provide finished rolls of precise dimension. Following slitting, the fabric is wound onto a larger role, either a full width role or a series of narrow slit rolls. The fabric rolls may further be wrapped and shipped.

In spunbond process, the fabrication of non-woven fabric includes the following steps: (a) extruding strands of the inventive polyethylene composition from a spinneret; (b) quenching the strands of the inventive polyethylene composition with a flow of air which is generally cooled in order to hasten the solidification of the molten strands of the inventive polyethylene composition; (c) attenuating the filaments by advancing them through the quench zone with a draw tension that can be applied by either pneumatically entraining the filaments in an air stream or by wrapping them around mechanical draw rolls of the type commonly used in the textile fibers industry; (d) collecting the drawn strands into a web on a foraminous surface, e.g. moving screen or porous belt; and (e) bonding the web of loose strands into the non-woven fabric. Bonding can be achieved by a variety of means including, but not limited to, thermo-calendering process, adhesive bonding process, hot air bonding process, needle punch process, hydroentangling process, and combinations thereof.

The inventive fabrics may have a tensile strength (MD) in the range of from 20 to 60 N/5 cm; for example, from 20 to 50 [1 N/5 cm; or in the alternative, from 25 to 50 N/5 cm; or in the alternative, from 30 to 50 N/5 cm; or in the alternative, from 30 to 60 N/5 cm; or in the alternative, from 25 to 60 N/5 cm. .

The inventive fabrics may have a tensile strength (CD) in the range of from 10 to 30 N/5 cm; for example, from 10 to 25 N/5 cm; or in the alternative, from 15 to 25 N/5 cm; or in the alternative, from 15 to 30 N/5 cm; or in the alternative, from 12 to 25 N/5 cm; or in the alternative, from 12 to 30 N/5 cm.

The inventive fabrics may have a tensile elongation (MD) in the range of from 50 to 200 percent; for example, from 50 to 150 percent; or in the alternative, from 75 to 200 percent; or in the alternative, from 75 to 150 percent; or in the alternative, from 100 to 200 percent ; or in the alternative, from 100 to 150 percent.

The inventive fabrics may have a tensile elongation (CD) in the range of from 50 to 250 percent; for example, from 75 to 250 percent; or in the alternative, from 100 to 250 percent; or in the alternative, from 50 to 200 percent; or in the alternative, from 60 to 250 percent; or in the alternative, from 60 to 250 percent.

The low levels of vinyl unsaturations in the inventive polyethylene composition are also important because such low levels of the vinyl unsaturations provide the instant inventive polyethylene composition with improved processability.

The inventive fabrics according to the instant invention may have an abrasion resistance in the range of less 1 mg/cm²; for example, in the range of from 0.2 to 0.5 mg/cm².

In one embodiment, the inventive spunbonded fabrics comprising bicomponent fibers having a core/sheath ratio of 80/20 to 40/60; for example, a core/sheath ratio of 80/20 to 40/60; or in the alternative, a core/sheath ratio of 70/30 to 40/60; or in the alternative, a core/sheath ratio of 75/25 to 40/60; or in the alternative, a core/sheath ratio of 70/30 to 50/50.

In another embodiment, the inventive spunbonded fabrics comprising bicomponent fibers having a fabric weight in a range of less than 75 g/m²; for example, less than 50 g/m²; or in the alternative, less than 40 g/m²; or in the alternative, less than 30 g/m²; or in the alternative, less than 30 g/m²; or in the alternative, less than 20 g/m²; or in the alternative, less than 15 g/m²; or in the alternative, less than 10 g/m².

The inventive polyethylene composition may be used in a variety of end-use applications including, but not limited to, carpet, apparel, upholstery, non-woven fabrics, woven fabrics, artificial turf, medical gowns, hospital wraps, and the like.

### EXAMPLES

The following examples illustrate the present invention but are not intended to limit the scope of the invention.

### Polyethylene Samples 1-2

### Catalyst Component Preparation

The hafnocene component can be prepared by techniques known in the art. For example, HfCl₄ (1.00 equiv.) can be added to ether at −30 to −50° C., and stirred to give a white suspension. The suspension can then be re-cooled to −30 to −50° C. , and then lithium propylcyclopentadienide (2.00 equiv.) added in portions. The reaction will turn light brown and become thick with suspended solid on adding the lithium propylcyclopentadienide. The reaction can then be allowed to warm slowly to room temperature and stirred for 10 to 20 hours. The resultant brown mixture can then be filtered to give brown solid and a straw yellow solution. The solid can then be washed with ether as is known in the art, and the combined ether solutions concentrated to under vacuum to give a cold, white suspension. Off-white solid product is then isolated by filtration and dried under vacuum, with yields of from 70 to 95 percent.

### Catalyst Composition Preparation

The catalyst compositions should be made at a Al/Hf mole ratio of from about 80:1 to 130:1 and the hafnium loading on the finished catalyst should be from about 0.6 to 0.8 weight percent Hf using the following general procedure. Methylaluminoxane (MAO) in toluene should be added to a clean, dry vessel and stirred at from 50 to 80 rpm and at a temperature in the range of 60 to 100° F. Additional toluene can then be added while stiffing. The hafnocene can then be dissolved in toluene and placed in the vessel with the MAO. The metallocene/MAO mixture can then be stirred at for from 30 min to 2 hours. Next, an appropriate amount of silica (average particle size in the range of 22 to 28 μm, dehydrated at 600° C.) can be added and stirred for another hour or more. The liquid can then be decanted and the catalyst composition dried at elevated temperature under flowing nitrogen while being stirred.

### Polymerization Process

The ethylene/1-hexene copolymers were produced in accordance with the following general procedure. The catalyst composition comprised a silica supported bis(n-propylcyclopentadienyl)hafnium dichloride with methalumoxane, the Al:Hf ratio being from about 80:1 to 130:1. The catalyst composition was injected dry into a fluidized bed gas phase polymerization reactor. More particularly, polymerization was conducted in a 336.5-419.3 mm ID diameter gas-phase fluidized bed reactor operating at approximately 2068 to 2586 kPa total pressure. The reactor bed weight was approximately 41-91 kg. Fluidizing gas was passed through the bed at a velocity of approximately 0.49 to 0.762 m per second. The fluidizing gas exiting the bed entered a resin disengaging zone located at the upper portion of the reactor. The fluidizing gas then entered a recycle loop and passed through a cycle gas compressor and water-cooled heat exchanger. The shell side water temperature was adjusted to maintain the reaction temperature to the specified value. Ethylene, hydrogen, 1-hexene and nitrogen were fed to the cycle gas loop just upstream of the compressor at quantities sufficient to maintain the desired gas concentrations. Gas concentrations were measured by an on-line vapor fraction analyzer. Product (the inventive polyethylene particles) was withdrawn from the reactor in batch mode into a purging vessel before it was transferred into a product bin. Residual catalyst and activator in the resin was deactivated in the product drum with a wet nitrogen purge. The catalyst was fed to the reactor bed through a stainless steel injection tube at a rate sufficient to maintain the desired polymer production rate. There were 2 separate polymerization runs conducted using this general process producing inventive polyethylene samples 1-2, as further described hereinbelow.

### Inventive Fibers and Fabrics 1-26

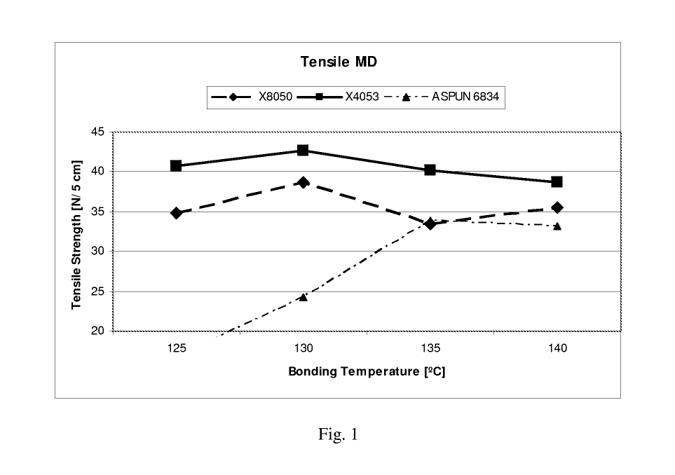

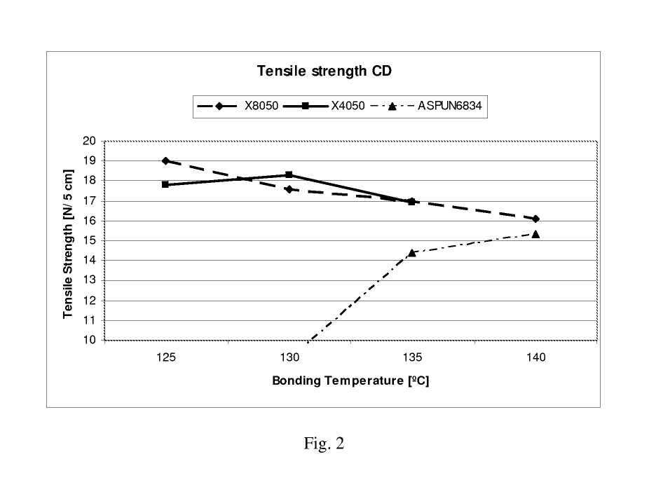

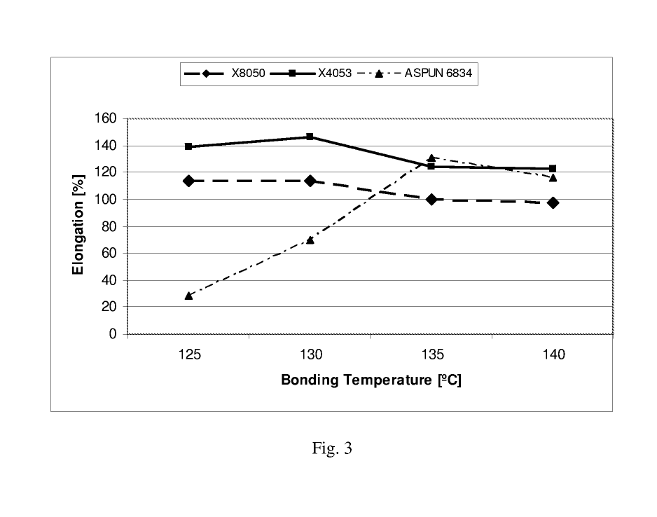

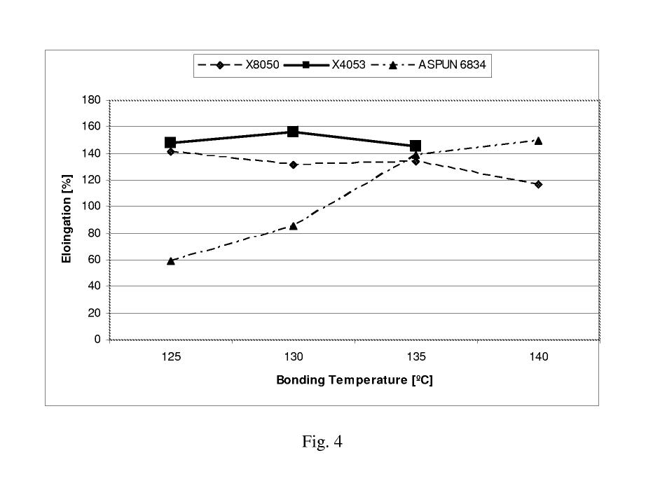

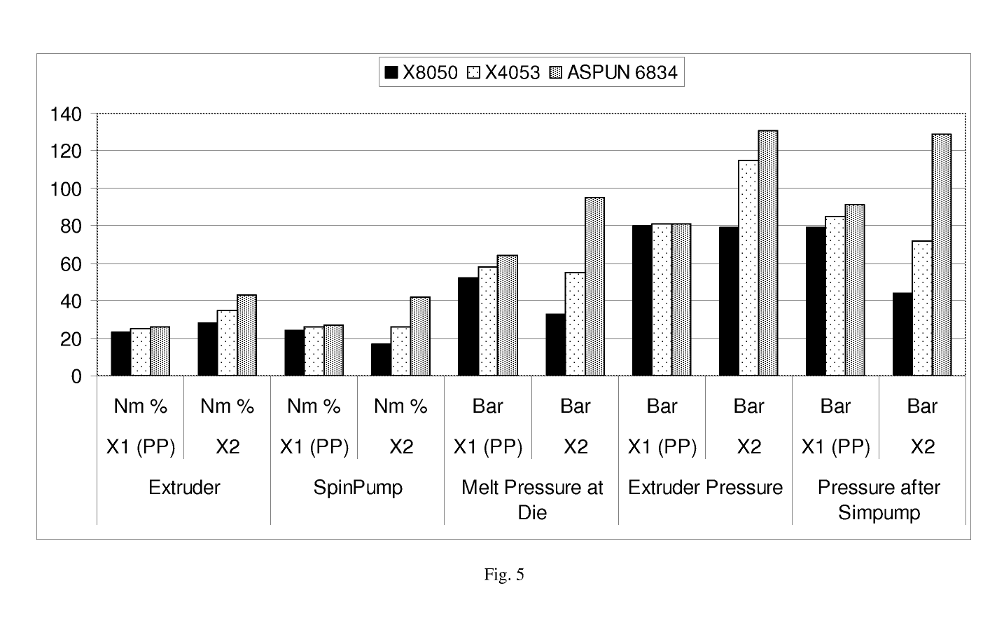

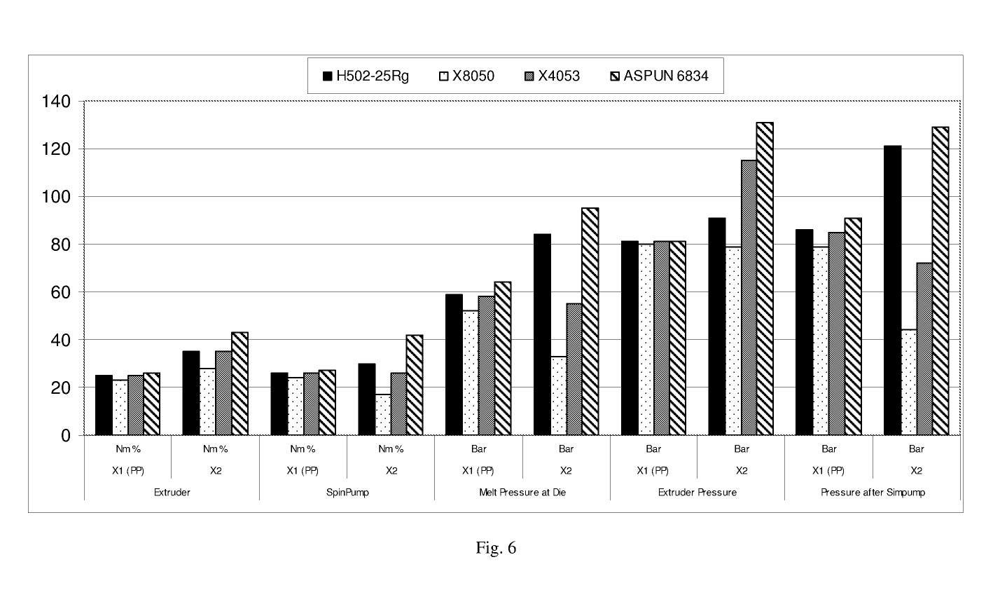

Inventive fibers 1-26 were prepared and then formed into inventive spunbond fabrics 1-26 according to the process described below, and tested for their physical properties. The results are shown in Table I, as well as FIGS. 1-6.

Inventive fibers 1-26 were produced via a Reicofil IV under the following conditions: (1) a die plate having 6300 holes per meter; (2) hole diameter of approximately 0.6 mm with LD ratio of 4; (3) line speed of approximately 175 m/minute; (4) output of approximately 240 kilogram/hours; (5) quench air temperature of approximately 18° C.; (6) cabin pressure of approximately 2800 Pa; (7) temperature of the spinneret of approximately 230 to 235° C.; (8) fabric weight of approximately 20 GSM; (9) calendar roll of approximate four different temperatures of 125, 130, 135, 140° C., respectively.

The extruded strands were quenched with a flow of air in order to hasten the solidification of the molten strands, and the filaments were attenuated by advancing them through the quench zone with a draw tension that was applied by either pneumatically entraining the filaments in an air stream or by wrapping them around mechanical draw rolls of the type commonly used in the textile fibers industry. The drawn strands were collected into a web on a foraminous surface, e.g. moving screen or porous belt, and bonded into a nonwoven fabric via thermo-calendering process, and combinations thereof.

### Comparative Fibers and Fabrics 1-13

Comparative fibers 1-13 were prepared and then formed into comparative spunbond fabrics 1-13 according to the process described below, and tested for their physical properties. The properties of the polymeric components of comparative fibers are ported in table IIA. The results are shown in Table II, as well as FIGS. 1-6.

Comparative fibers 1-12 produced via a Reincofil IV under the following conditions: (1) a die plate having 6300 holes per meter; (2) hole diameter of approximately 0.6 mm with LD ratio of 4; (3) line speed of approximately 175 m/minute; (4) output of approximately 240 kilogram/hours; (5) quench air temperature of approximately 18° C.; (6) cabin pressure of approximately 2800 Pa; (7) temperature of the spinneret of approximately 230 to 235° C.; (8) fabric weight of approximately 20 GSM; (9) calendar roll of approximate four different temperatures of 125, 130, 135, 140° C., respectively.

The extruded strands were quench with a flow of air in order to hasten the solidification of the molten strands, and the filaments were attenuated by advancing them through the quench zone with a draw tension that was applied by either pneumatically entraining the filaments in an air stream or by wrapping them around mechanical draw rolls of the type commonly used in the textile fibers industry. The drawn strands were collected into a web on a foraminous surface, e.g. moving screen or porous belt, and bonded into a nonwoven fabric via thermo-calendering process, and combinations thereof.

### Polymeric components used in Inventive Examples and Comparative Examples

Inventive polyethylene sample 1 (X8050) is a polyethylene composition having a melt index (I₂), measured at 190° C. and 2.16 kg, of approximately 80 g/10 minutes and a density of approximately 0.955 g/cm³.
Inventive polyethylene sample 2 (X4053) is a polyethylene composition having a melt index (I₂), measured at 190° C. and 2.16 kg, of approximately 40 g/10 minutes and a density of approximately 0.955 g/cm³.
Comparative polyethylene sample 1 (ASPUN 6834) is a polyethylene composition (ethylene octane copolymer) having a melt index (I₂) of approximately 17 g/10 minutes and a density of approximately 0.950 g/cm³.
PP Standard (HSO2-25RG) is a propylene ethylene copolymer having a melt flow rate, measured at 200° C. and 2.16 kg, in the range of 23.5 to 25.5 g/10 minutes.

### Test Methods

Test methods include the following:

Density (g/cm³) was measured according to ASTM-D 792-03, Method B, in isopropanol. Specimens were measured within 1 hour of molding after conditioning in the isopropanol bath at 23° C. for 8 min to achieve thermal equilibrium prior to measurement. The specimens were compression molded according to ASTM D-4703-00 Annex A with a 5 min initial heating period at about 190° C. and a 15° C./min cooling rate per Procedure C. The specimen was cooled to 45° C. in the press with continued cooling until “cool to the touch.”

Melt index (I₂) was measured at 190° C. under a load of 2.16 kg according to ASTM D-1238-03.

Weight average molecular weight (M_{w}) and number average molecular weight (Mₙ) were determined according to methods known in the art using triple detector GPC, as described herein below.

The molecular weight distributions of the ethylene polymers were determined by gel permeation chromatography (GPC). The chromatographic system consisted of a Waters (Millford, Mass.) 150° C. high temperature gel permeation chromatograph, equipped with a Precision Detectors (Amherst, Mass.) 2-angle laser light scattering detector Model 2040. The 15° angle of the light scattering detector was used for calculation purposes. Data collection was performed using Viscotek TriSEC software version 3 and a 4-channel Viscotek Data Manager DM400. The system was equipped with an on-line solvent degas device from Polymer Laboratories. The carousel compartment was operated at 140° C. and the column compartment was operated at 150° C. The columns used were four Shodex HT 806M 300 mm, 13 μm columns and one Shodex HT803M 150 mm, 12 μm column. The solvent used was 1,2,4 trichlorobenzene. The samples were prepared at a concentration of 0.1 grams of polymer in 50 milliliters of solvent. The chromatographic solvent and the sample preparation solvent contained 200 μg/g of butylated hydroxytoluene (BHT). Both solvent sources were nitrogen sparged. Polyethylene samples were stirred gently at 160° C. for 4 hours. The injection volume used was 200 microliters, and the flow rate was 0.67 milliliters/min. Calibration of the GPC column set was performed with 21 narrow molecular weight distribution polystyrene standards, with molecular weights ranging from 580 to 8,400,000 g/mol, which were arranged in 6 “cocktail” mixtures with at least a decade of separation between individual molecular weights. The standards were purchased from Polymer Laboratories (Shropshire, UK). The polystyrene standards were prepared at 0.025 grams in 50 milliliters of solvent for molecular weights equal to, or greater than, 1,000,000 g/mol, and 0.05 grams in 50 milliliters of solvent for molecular weights less than 1,000,000 g/mol. The polystyrene standards were dissolved at 80° C. with gentle agitation for 30 minutes. The narrow standards mixtures were run first, and in order of decreasing highest molecular weight component, to minimize degradation. The polystyrene standard peak molecular weights were converted to polyethylene molecular weights using the following equation (as described in Williams and Ward, J. Polym. Sci., Polym. Let., 6, 621 (1968)):

Mpolyethylene=A×(Mpolystyrene)^{B},

where M is the molecular weight, A has a value of 0.41 and B is equal to 1.0. The Systematic Approach for the determination of multi-detector offsets was done in a manner consistent with that published by Balke, Mourey, et al. (Mourey and Balke, Chromatography Polym. Chpt 12, (1992) and Balke, Thitiratsakul, Lew, Cheung, Mourey, Chromatography Polym. Chpt 13, (1992)), optimizing dual detector log results from Dow broad polystyrene 1683 to the narrow standard column calibration results from the narrow standards calibration curve using in-house software. The molecular weight data for off-set determination was obtained in a manner consistent with that published by Zimm (Zimm, B. H., J. Chem. Phys., 16, 1099 (1948)) and Kratochvil (Kratochvil, P., Classical Light Scattering from Polymer Solutions, Elsevier, Oxford, N.Y. (1987)). The overall injected concentration used for the determination of the molecular weight was obtained from the sample refractive index area and the refractive index detector calibration from a linear polyethylene homopolymer of 115,000 g/mol molecular weight, which was measured in reference to NIST polyethylene homopolymer standard 1475. The chromatographic concentrations were assumed low enough to eliminate addressing 2^{nd} Virial coefficient effects (concentration effects on molecular weight). Molecular weight calculations were performed using in-house software. The calculation of the number-average molecular weight, weight-average molecular weight, and z-average molecular weight were made according to the following equations, assuming that the refractometer signal is directly proportional to weight fraction. The baseline-subtracted refractometer signal can be directly substituted for weight fraction in the equations below. Note that the molecular weight can be from the conventional calibration curve or the absolute molecular weight from the light scattering to refractometer ratio. An improved estimation of z-average molecular weight, the baseline-subtracted light scattering signal can be substituted for the product of weight average molecular weight and weight fraction in equation (2) below:

$$a ) \overline{Mn} = \frac{{\sum }^{i} {Wf}_{i}}{{\sum }^{i} ( {Wf}_{i} / {M}_{i} )} \\ b ) \overline{Mw} = \frac{{\sum }^{i} ( {Wf}_{i} * {M}_{i} )}{{\sum }^{i} {Wf}_{i}} \\ c ) \overline{Mz} = \frac{{\sum }^{i} ( {Wf}_{i} * {M}_{i}^{2} )}{{\sum }^{i} ( {Wf}_{i} * {M}_{i} )} \qquad ( 2 )$$

Monomodal distribution was characterized according to the weight fraction of the highest temperature peak in temperature rising elution fractionation (typically abbreviated as “TREF”) data as described, for example, in Wild et al., Journal of Polymer Science, Poly. Phys. Ed., Vol. 20, p. 441 (1982), in U.S. Pat. No. 4,798,081 (Hazlitt et al.), or in U.S. Pat. No. 5,089,321 (Chum et al.), the disclosures of all of which are incorporated herein by reference. In analytical temperature rising elution fractionation analysis (as described in U.S. Pat. No. 4,798,081 and abbreviated herein as “ATREF”), the composition to be analyzed is dissolved in a suitable hot solvent (for example, 1,2,4 trichlorobenzene), and allowed to crystallized in a column containing an inert support (for example, stainless steel shot) by slowly reducing the temperature. The column was equipped with both an infra-red detector and a differential viscometer (DV) detector. An ATREF-DV chromatogram curve was then generated by eluting the crystallized polymer sample from the column by slowly increasing the temperature of the eluting solvent (1,2,4 trichlorobenzene). The ATREF-DV method is described in further detail in WO 99/14271, the disclosure of which is incorporated herein by reference.

Long Chain Branching was determined according to the methods known in the art, such as gel permeation chromatography coupled with low angle laser light scattering detector (GPC-LALLS) and gel permeation chromatography coupled with a differential viscometer detector (GPC-DV).

Short chain branch distribution breadth (SCBDB) was determined based in the data obtained via analytical temperature rising elution fractionation (ATREF) analysis, described hereinbelow in further details. First, a cumulative distribution of the elution curve was calculated beginning at 30° C. and continuing to and including 109° C. From the cumulative distribution, temperatures were selected at 5 weight percent (T₅) and 95 weight percent (T₉₅). These two temperatures were then used as the bounds for the SCBDB calculation. The SCBDB is then calculated from the following equation:

$$SCBDB = \sqrt{\frac{{\sum }_{i} {{w}_{i} ( {T}_{i} - {T}_{w} )}^{2}}{{\sum }_{i} {w}_{i}}}$$

for all Tᵢ including and between T₅ and T₉₅. Tᵢ is the temperature at the ith point on the elution curve, wᵢ is the weight fraction of material from each temperature on the elution curve, and T_{w} is the weight-averaged temperature of the elution curve (Σ(wᵢTᵢ)/Σwᵢ) between and including T₅ and T₉₅.

Analytical temperature rising elution fractionation (ATREF) analysis was conducted according to the method described in U.S. Pat. No. 4,798,081 and Wilde, L.; Ryle, T. R.; Knobeloch, D. C.; Peat, I. R.; Determination of Branching Distributions in Polyethylene and Ethylene Copolymers, J. Polym. Sci., 20, 441-455 (1982), which are incorporated by reference herein in their entirety. The composition to be analyzed was dissolved in trichlorobenzene and allowed to crystallize in a column containing an inert support (stainless steel shot) by slowly reducing the temperature to 20° C. at a cooling rate of 0.1° C./min. The column was equipped with an infrared detector. An ATREF chromatogram curve was then generated by eluting the crystallized polymer sample from the column by slowly increasing the temperature of the eluting solvent (trichlorobenzene) from 20 to 120° C. at a rate of 1.5° C./min.

Comonomer content was measured using C₁₃ NMR, as discussed in Randall, Rev. Macromol. Chem. Chys., C29 (2&3), pp. 285-297, and in U.S. Pat. No. 5,292,845, the disclosures of which are incorporated herein by reference to the extent related to such measurement. The samples were prepared by adding approximately 3 g of a 50/50 mixture of tetrachloroethane-d2/orthodichlorobenzene that was 0.025M in chromium acetylacetonate (relaxation agent) to 0.4 g sample in a 10 mm NMR tube. The samples were dissolved and homogenized by heating the tube and its contents to 150° C. The data was collected using a JEOL Eclipse 400 MHz NMR spectrometer, corresponding to a 13 C resonance frequency of 100.6 MHz. Acquisition parameters were selected to ensure quantitative 13 C data acquisition in the presence of the relaxation agent. The data was acquired using gated 1H decoupling, 4000 transients per data file, a 4.7 sec relaxation delay and 1.3 second acquisition time, a spectral width of 24,200 Hz and a file size of 64K data points, with the probe head heated to 130° C. The spectra were referenced to the methylene peak at 30 ppm. The results were calculated according to ASTM method D5017-91.

Melt temperature and crystallization temperature were measured via Differential Scanning calorimetry (DSC). All of the results reported here were generated via a TA Instruments Model Q1000 DSC equipped with an RCS (refrigerated cooling system) cooling accessory and an auto sampler. A nitrogen purge gas flow of 50 ml/min was used throughout. The sample was pressed into a thin film using a press at 175° C. and 1500 psi (10.3 MPa) maximum pressure for about 15 seconds, then air-cooled to room temperature at atmospheric pressure. About 3 to 10 mg of material was then cut into a 6 mm diameter disk using a paper hole punch, weighed to the nearest 0.001 mg, placed in a light aluminum pan (ca 50 mg) and then crimped shut. The thermal behavior of the sample was investigated with the following temperature profile: The sample was rapidly heated to 180° C. and held isothermal for 3 minutes in order to remove any previous thermal history. The sample was then cooled to −40° C. at 10° C./min cooling rate and was held at −40° C. for 3 minutes. The sample was then heated to 150° C. at 10° C./min heating rate. The cooling and second heating curves were recorded.

Vinyl unsaturations were measured according to ASTM D-6248-98.

Abrasion resistance was measured by abrading a spunbond fabric using a Sutherland 2000 Rub Tester to determine the fuzz level. An 11.0 cm×4.0 cm piece of non-woven spunbond fabric was abraded with 320-grit aluminum oxide sandpaper under 2 lbs weight with 20 cycles at a rate of 42 cycles per minute, which resulted in loose fibers accumulating on the top of the spunbond fabric. The loose fibers were collected using tape and measured gravimetrically.

Tensile Elongation in machine direction (MD) in the range of from 50 to 150 percent, measured by cutting Spunbond fabrics into 1×6 inch specimens and testing the specimens in the machine direction (MD) using an INSTRON. The specimens were tested at 8 inches/minute with 4 inches gauge. The MD extensibility was determined at the peak force.

Tensile Elongation in cross direction (CD) in the range of from 50 to 250 percent, measured by cutting Spunbond fabrics into 1×6 inch specimens and testing the specimens in the cross direction (CD) using an INSTRON. The specimens were tested at 8 inches/minute with 4 inches gauge. The CD extensibility was determined at the peak force.

Tensile Strength in machine direction (MD) in the range of from 20 to 60 N/5 cm, measured by cutting Spunbond fabrics into 1×6 inch specimens and testing the specimens in the machine direction (MD) using an INSTRON. The specimens were tested at 8 inches/minute with 4 inches gauge. The MD tensile strength was determined at the peak force

Tensile Strength in cross direction (CD) in the range of from 10 to 30 N/5 cm, measured by cutting Spunbond fabrics into 1×6 inch specimens and testing the specimens in the cross direction (CD) using an INSTRON. The specimens were tested at 8 inches/minute with 4 inches gauge. The CD tensile strength was determined at the peak force.

The present invention may be embodied in other forms without departing from the spirit and the essential attributes thereof, and, accordingly, reference should be made to the appended claims, rather than to the foregoing specification, as indicating the scope of the invention.

TABLE I

| Inventive | | | | | | Calendar | Tensile | Tensile | Elongation |
|---|---|---|---|---|---|---|---|---|---|
| Sample | 15 | 20 | Core/Sheath | | | SET | MD | CD | MD |
| Number | gsm | gsm | ratio | Core | Sheath | (° C.) | (N/5 cm) | (N/5 cm) | (%) |
| 1 | 330 | 329 | 50/50 | Standard | Inventive | 125 | 34.9 | 19 | 113.5 |
| | | | | PP | PE 1 | | | | |
| 2 | 332 | 331 | 50/50 | Standard | Inventive | 130 | 38.7 | 17.6 | 113.5 |
| | | | | PP | PE 1 | | | | |
| 3 | 342 | 341 | 50/50 | Standard | Inventive | 135 | 33.4 | 17.0 | 100.3 |
| | | | | PP | PE 1 | | | | |
| 4 | 344 | 343 | 50/50 | Standard | Inventive | 140 | 35.5 | 16.1 | 97.4 |
| | | | | PP | PE 1 | | | | |
| 5 | 326 | 325 | 70/30 | Standard | Inventive | 125 | — | — | — |
| | | | | PP | PE 1 | | | | |
| 6 | 224 | 333 | 70/30 | Standard | Inventive | 130 | — | — | — |
| | | | | PP | PE 1 | | | | |
| 7 | 340 | 339 | 70/30 | Standard | Inventive | 135 | — | — | — |
| | | | | PP | PE 1 | | | | |
| 8 | 346 | 345 | 70/30 | Standard | Inventive | 140 | — | — | — |
| | | | | PP | PE 1 | | | | |
| 9 | 328 | 327 | 90/10 | Standard | Inventive | 125 | — | — | — |
| | | | | PP | PE 1 | | | | |
| 10 | 226 | 335 | 90/10 | Standard | Inventive | 130 | — | — | — |
| | | | | PP | PE 1 | | | | |
| 11 | 338 | 337 | 90/10 | Standard | Inventive | 135 | — | — | — |
| | | | | PP | PE 1 | | | | |
| 12 | 348 | 347 | 90/10 | Standard | Inventive | 140 | — | — | — |
| | | | | PP | PE 1 | | | | |
| 13 | | | 50/50 | Standard | Inventive | 140 | — | — | — |
| | | | | PP | PE 1 | | | | |
| 14 | 350 | 349 | 50/50 | Standard | Inventive | 125 | 40.7 | 17.8 | 138.8 |
| | | | | PP | PE 2 | | | | |
| 15 | 360 | 359 | 50/50 | Standard | Inventive | 130 | 42.7 | 18.3 | 145.9 |
| | | | | PP | PE 2 | | | | |
| 16 | 362 | 361 | 50/50 | Standard | Inventive | 135 | 40.2 | 16.9 | 124.3 |
| | | | | PP | PE 2 | | | | |
| 17 | 372 | 371 | 50/50 | Standard | Inventive | 140 | 38.7 | — | 122.9 |
| | | | | PP | PE 2 | | | | |
| 18 | 352 | 351 | 70/30 | Standard | Inventive | 125 | — | — | — |
| | | | | PP | PE 2 | | | | |
| 19 | 358 | 357 | 70/30 | Standard | Inventive | 130 | — | — | — |
| | | | | PP | PE 2 | | | | |
| 20 | 364 | 363 | 70/30 | Standard | Inventive | 135 | — | — | — |
| | | | | PP | PE 2 | | | | |
| 21 | 370 | 369 | 70/30 | Standard | Inventive | 140 | — | — | — |
| | | | | PP | PE 2 | | | | |
| 22 | 354 | 353 | 90/10 | Standard | Inventive | 125 | — | — | — |
| | | | | PP | PE 2 | | | | |
| 23 | 356 | 355 | 90/10 | Standard | Inventive | 130 | — | — | — |
| | | | | PP | PE 2 | | | | |
| 24 | 366 | 365 | 90/10 | Standard | Inventive | 135 | — | — | — |
| | | | | PP | PE 2 | | | | |
| 25 | 368 | 367 | 90/10 | Standard | Inventive | 140 | — | — | — |
| | | | | PP | PE 2 | | | | |
| 26 | | 373 | 50/50 | Standard | Inventive | 140 | — | — | — |
| | | | | PP | PE 2 | | | | |

TABLE II

| | | | | | | Calendar | Tensile | Tensile | Elongation |
|---|---|---|---|---|---|---|---|---|---|
| Comparative | 15 | 20 | Core/Sheath | | | SET | MD | CD | MD |
| Sample No. | gsm | gsm | ratio | Core | Sheath | (° C.) | (N/5 cm) | (N/5 cm) | (%) |
| 1 | 379 | 378 | 50/50 | Standard | Comparative | 125 | — | — | — |
| | | | | PP | PE 1 | | | | |
| 2 | 381 | 380 | 50/50 | Standard | Comparative | 130 | — | — | — |
| | | | | PP | PE 1 | | | | |
| 3 | 391 | 390 | 50/50 | Standard | Comparative | 135 | 33.9 | 14.37 | 130.9 |
| | | | | PP | PE 1 | | | | |
| 4 | 393 | 392 | 50/50 | Standard | Comparative | 140 | — | — | — |
| | | | | PP | PE 1 | | | | |
| 5 | 377 | 376 | 70/30 | Standard | Comparative | 125 | — | — | — |
| | | | | PP | PE 1 | | | | |
| 6 | 383 | 382 | 70/30 | Standard | Comparative | 130 | — | — | — |
| | | | | PP | PE 1 | | | | |
| 7 | 389 | 388 | 70/30 | Standard | Comparative | 135 | — | — | — |
| | | | | PP | PE 1 | | | | |
| 8 | 395 | 394 | 70/30 | Standard | Comparative | 140 | — | — | — |
| | | | | PP | PE 1 | | | | |
| 9 | 375 | 374 | 90/10 | Standard | Comparative | 125 | — | — | — |
| | | | | PP | PE 1 | | | | |
| 10 | 385 | 384 | 90/10 | Standard | Comparative | 130 | — | — | — |
| | | | | PP | PE 1 | | | | |
| 11 | 387 | 386 | 90/10 | Standard | Comparative | 135 | — | — | — |
| | | | | PP | PE 1 | | | | |
| 12 | 397 | 396 | 90/10 | Standard | Comparative | 140 | — | — | — |
| | | | | PP | PE 1 | | | | |
| 13 | | 398 | 50/50 | Standard | Comparative | 140 | — | — | — |
| | | | | PP | PE 1 | | | | |

We claim:

1. A bicomponent fiber comprising:
a first component comprising a polymeric material selected from the group consisting of polypropylene, polyester, and polyamide; and
a second component comprising a polyethylene composition comprising:
less than or equal to 100 percent by weight of the units derived from ethylene;
less than 20 percent by weight of units derived from one or more α-olefin comonomers;
wherein said polyethylene composition has a density in the range of from 0.945 to 0.965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition.

2. The bicomponent fiber according to claim 1, wherein said fiber has a denier per filament in the range of from 1.6 to 2.4 g/9000 m.

3. The bicomponent fiber according to claim 1, wherein said bicomponent fiber has a sheath-core configuration, and wherein the core comprises the first component and the sheath comprises the second component.

4. The bicomponent fiber of claim 3, wherein said bicomponent fiber is a core/sheath configuration and has an 80/20 to 40/60 core/sheath ratio.

5. The bicomponent fiber according to claim 1, wherein said bicomponent fiber is a continuous fiber or a staple fiber.

6. A process for producing a bicomponent fiber comprising the steps of:
selecting a first component comprising a polymeric material selected from the group consisting of polypropylene, polyester, and polyamide; and
selecting a second component comprising a polyethylene composition comprising:
less than or equal to 100 percent by weight of the units derived from ethylene;
less than 20 percent by weight of units derived from one or more α-olefin comonomers;
wherein said polyethylene composition has a density in the range of from 0.945 to 0.965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition;
spinning said first component and said second component into a bicomponent fiber; and
thereby forming said bicomponent fiber.

7. The process for producing a bicomponent fiber according to claim 6, wherein said process further comprises the step of orienting said fiber.

8. The process for producing a bicomponent fiber according to claim 7, wherein said fiber is oriented via cold drawing.

9. The process for producing a bicomponent fiber according to claim 6, wherein process further comprises the step of annealing said fiber.

10. The process for producing a bicomponent fiber according to claim 11, wherein said annealing step is carried out at 100° C. or above.

11. The process for producing a bicomponent fiber according to claim 10, wherein said fiber is annealed at a fixed length.

12. A process for fabricating a spunbond fabric comprising the steps of:
selecting a first component comprising a polymeric material selected from the group consisting of polypropylene, polyester, and polyamide; and
selecting a second component comprising a polyethylene composition comprising:
less than or equal to 100 percent by weight of the units derived from ethylene;
less than 20 percent by weight of units derived from one or more α-olefin comonomers;
wherein said polyethylene composition has a density in the range of from 0.945 to 0.965 g/cm³, a molecular weight distribution (M_{w}/Mₙ) in the range of from 1.70 to 3.5, a melt index (I₂) in the range of from 0.2 to 150 g/10 minutes, a molecular weight distribution (M_{z}/M_{w}) in the range of from less than 2.5, vinyl unsaturation in the range of from less than 0.1 vinyls per one thousand carbon atoms present in the backbone of said composition;
spinning said first component and said second component into one or more bicomponent fibers;
disposing said one or more bicomponent fiber on a surface;
thereby forming a web:
bonding said one or more bicomponent fibers in said web;
thereby forming said spunbond fabric.

13. A nonwoven fabric comprising one or more bicomponent fibers according to claim 1.

14. The nonwoven fabric according to claim 13, wherein said nonwoven fabric has an abrasion resistance in the range of from less than 1 mg/cm².

15. The nonwoven fabric according to claim 13, wherein said fabric has a tensile elongation in the machine direction in the range of from 50 to 200 percent and a tensile elongation in the cross direction in the range of from 50 to 250 percent.

16. An article comprising one or more nonwoven fabrics according to claim 13.

17. The article according to claim 15, wherein said article is selected from the group consisting of upholstery, apparel, wall covering, carpet, diaper topsheet, diaper backsheet, medical fabric, surgical wrap, hospital gown, wipe, textile, and geotextile.
